(19) United States
(12) Patent Application Publication
(10) Pub. No.: US 2025/0102332 A1
(43) Pub. Date: Mar. 27, 2025

(54) MEASURING DEVICE AND MEASURING SYSTEM

(71) Applicant: HORIBA Advanced Techno, Co., Ltd., Kyoto-shi, Kyoto (JP)

(72) Inventors: Issei KOBAYASHI, Kyoto-shi (JP); Wataru KINOSHITA, Kyoto-shi (JP); Kentaro INOUE, Kyoto-shi (JP); Akihiro HASEGAWA, Tokyo (JP)

(21) Appl. No.: 18/727,898

(22) PCT Filed: Feb. 1, 2023
(86) PCT No.: PCT/JP2023/003182

§ 371 Date: Jul. 10, 2024

(30) Foreign Application Priority Data
Feb. 1, 2022 (JP) 2022-013955

(51) Int. Cl. G01D 11/30

(52) U.S. Cl. CPC G01D 11/30

(57) ABSTRACT

At a measuring device, a measurement gauge comprises a measurement fluid passage and a measurement connection port, a main body comprises a main body fluid passage and a main body connection port, and a connector comprises a connecting tube, a first end of which is inserted into an interior of the measurement connection port, and a second end of which is inserted into an interior of the main body connection port, an annular first elastic body which seals a region between an outside circumferential portion of the first end of the connecting tube and an inside circumferential portion of the measurement connection port, and an annular second elastic body which seals a region between an outside circumferential portion of the second end of the connecting tube and an inside circumferential portion of the main body connection port.

### TECHNICAL FIELD

This application relates to a measuring device and a measuring system.

### BACKGROUND ART

Conventionally, a measuring device might, for example, comprise a measurement gauge that measures a fluid, and a main body with respect to which the measurement gauge is removably installable (e.g., Patent Reference No. 1). Measurement gauges comprise measurement fluid passages through which fluid flows; main bodies comprise main body fluid passages through which fluid flows.

In addition, when measurement gauges are attached to main bodies, measurement fluid passages and main body fluid passages are connected. It may so happen, for example, where an attachment error, manufacturing error, or the like exists, and the location of a measurement fluid passage with respect to a main body fluid passage is misaligned relative to the reference location therefor, that there is a possibility that the connection between the measurement fluid passage and the main body fluid passage will be inadequate. In such case, there is a possibility, for example, that the fluid may leak from the fluid passage.

### CITATION LIST

### Patent Literature

Patent Reference No. 1: US A 2014/312609

### SUMMARY OF THE INVENTION

### Technical Problem

The problem is therefore to provide a measuring device and measuring system that will make it possible to definitively connect measurement fluid passage(s) and main body fluid passage(s).

### Solution to Problem

There is provided a measuring device comprising:
a measurement gauge which measures a fluid;
a main body with respect to which the measurement gauge is removably installable; and
a connector which connects the measurement gauge and the main body;
wherein the measurement gauge comprises
   a measurement fluid passage through which the fluid flows, and
   a recessed measurement connection port which is coupled to an end of the measurement fluid passage;
wherein the main body comprises
   a main body fluid passage through which the fluid flows, and
   a recessed main body connection port which is coupled to an end of the main body fluid passage; and
wherein the connector comprises
   a connecting tube, a first end of which is inserted into an interior of the measurement connection port, and a second end of which is inserted into an interior of the main body connection port;
   an annular first elastic body which seals a region between an outside circumferential portion of the first end of the connecting tube and an inside circumferential portion of the measurement connection port; and
   an annular second elastic body which seals a region between an outside circumferential portion of the second end of the connecting tube and an inside circumferential portion of the main body connection port.

There is provided a measuring system comprising:
the measuring device; and
a communication apparatus that is capable of communicating with the measuring device.

### BRIEF DESCRIPTION OF THE DRAWINGS

FIG. 1 is a schematic overview of a measuring system associated with an embodiment.

FIG. 2 is a schematic overview of a measuring system at same embodiment, being a drawing showing a situation such as might exist when one measurement gauge is detached from the main body.

FIG. 3 is a schematic diagram showing fluid passages at a measuring device associated with same embodiment.

FIG. 4 is a block diagram of control which may occur at a measuring system associated with same embodiment.

FIG. 5 is a block diagram of control which may occur at a measuring system associated with same embodiment.

FIG. 6 is a side view of a measuring device associated with same embodiment.

FIG. 7 is an enlarged sectional view of the principal components in a section taken along VII-VII in FIG. 6.

FIG. 8 is an enlarged view of the principal components in FIG. 7.

FIG. 9 is a perspective view of the principal components as seen from below a measurement gauge associated with same embodiment.

FIG. 10 is a drawing of the principal components at a measurement gauge associated with same embodiment, a part of which is a side view showing a section thereof.

FIG. 11 is an enlarged view of the principal components in a section taken along XI-XI in FIG. 10.

FIG. 12 is a perspective view of the principal components at a main body associated with same embodiment.

FIG. 13 is an enlarged sectional view of the principal components in a section taken along XIII-XIII in FIG. 12.

FIG. 14 is a drawing of the principal components at a main body associated with same embodiment, a part of which is a side view of a section taken along XIV-XIV in FIG. 13.

FIG. 15 is a perspective view showing the interior of a main body associated with same embodiment.

FIG. 16 is a perspective view showing the interior of a main body associated with same embodiment.

FIG. 17 is a side view showing how actuation might proceed from the state at FIG. 14.

FIG. 18 is a drawing of a situation that might exist before a measurement gauge is attached to a main body, a part of which is a side view showing a section thereof.

FIG. 19 is a drawing showing a situation in which the main body has moved downward from the state at FIG. 18.

FIG. 20 is a drawing showing a situation in which the main body has moved downward from the state at FIG. 19, and the measurement gauge has been attached to the main body.

FIG. 21 is a longitudinal sectional view of principal components showing a situation that might exist when a measurement gauge has been attached to a main body.

FIG. 22 is an enlarged view of region XXII in FIG. 19.

FIG. 23 is an enlarged view of region XXIII in FIG. 20.

FIG. 24 is an enlarged view of region XXIV in FIG. 20.

FIG. 25 is a drawing of a situation that might exist when a measurement gauge is detached from a main body, being a side view showing a partial section thereof.

FIG. 26 is a perspective view of a measuring device associated with another embodiment.

FIG. 27 is a perspective view of a measuring device associated with same embodiment, being a drawing showing a situation such as might exist when the measurement gauge is detached from the main body.

FIG. 28 is a perspective view of a main body associated with same embodiment.

FIG. 29 is a side view showing a partial section of a measuring device associated with same embodiment.

FIG. 30 is a side view showing a partial section of a measuring device associated with same embodiment, being a drawing showing a situation such as might exist when the measurement gauge is removably installed with respect to the main body.

FIG. 31 is a side view showing a partial section of a measuring device associated with same embodiment, being a drawing showing a situation such as might exist when the measurement gauge is removably installed with respect to the main body.

### DESCRIPTION OF EMBODIMENTS

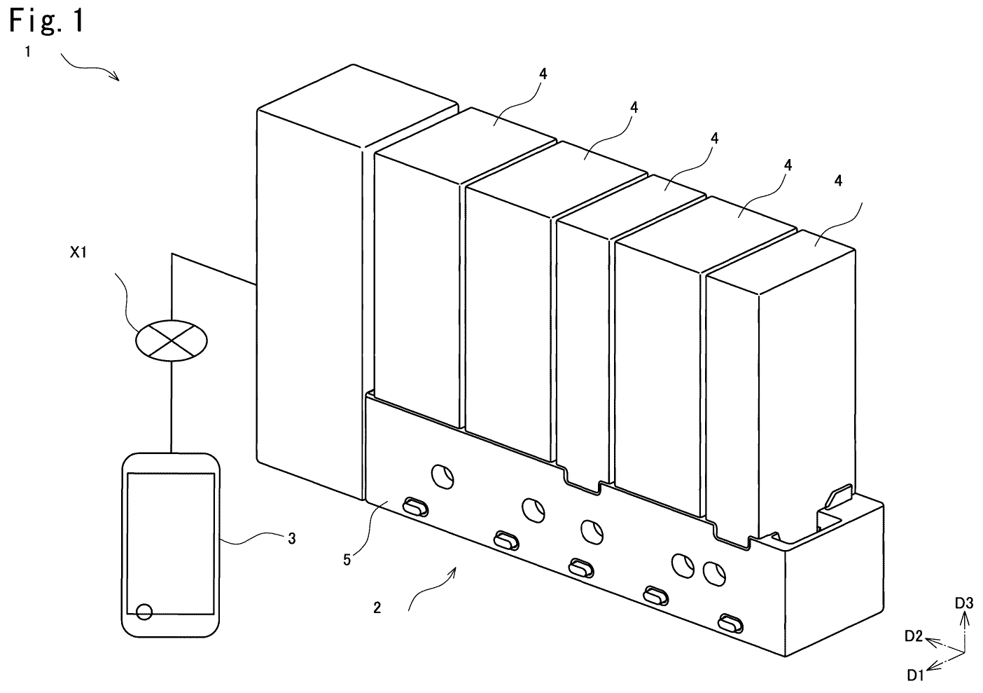

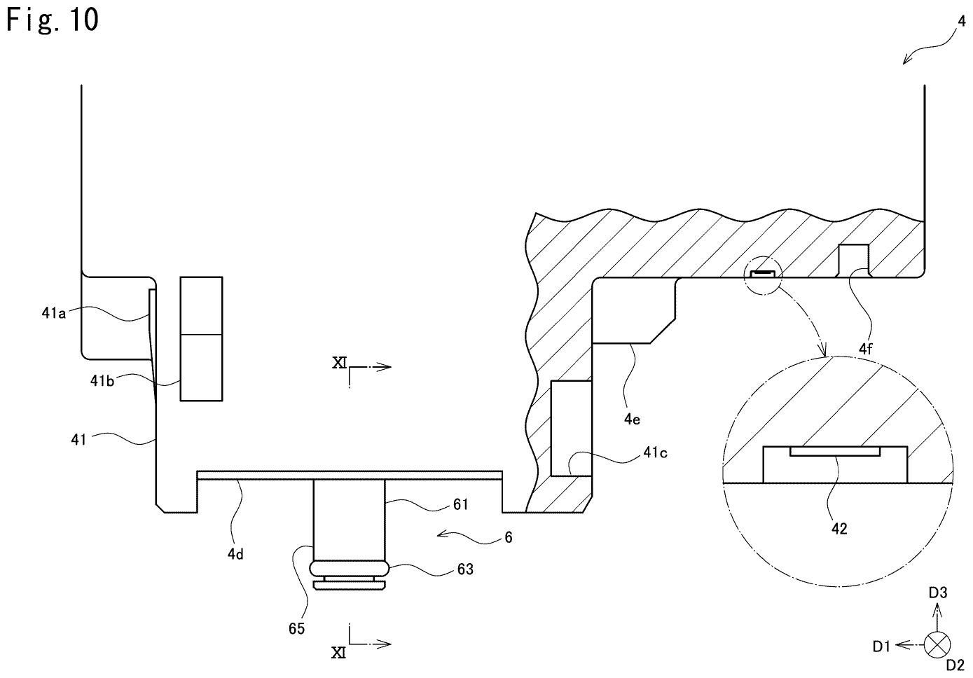

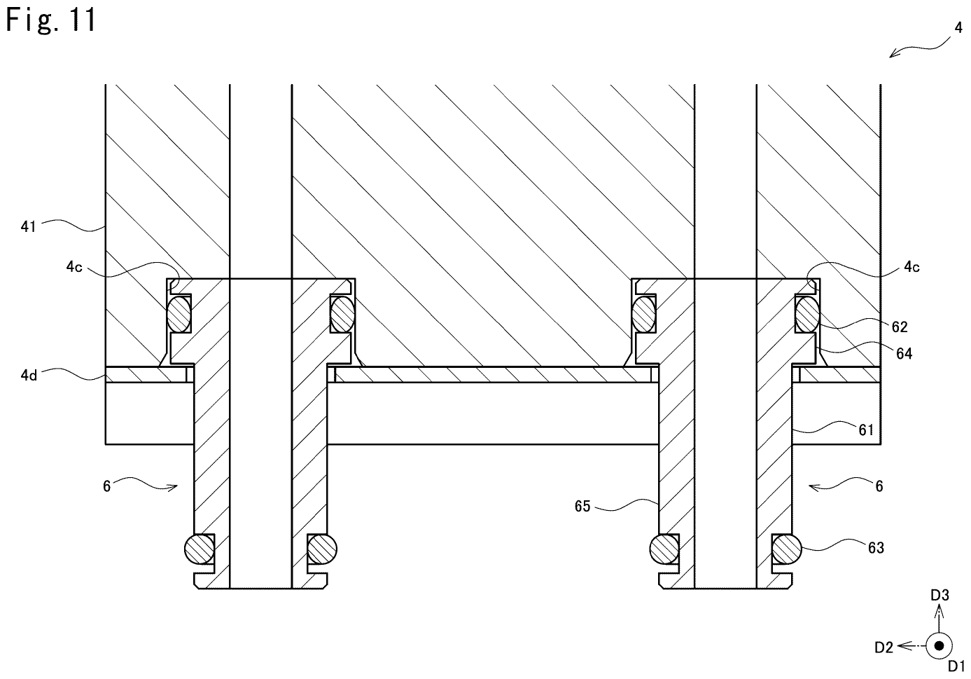

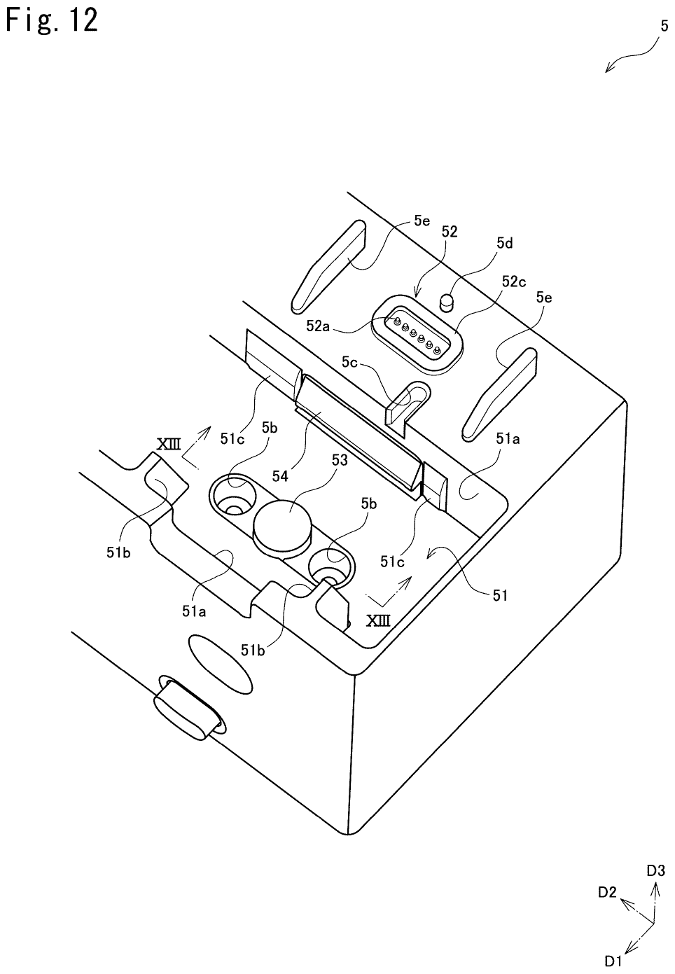

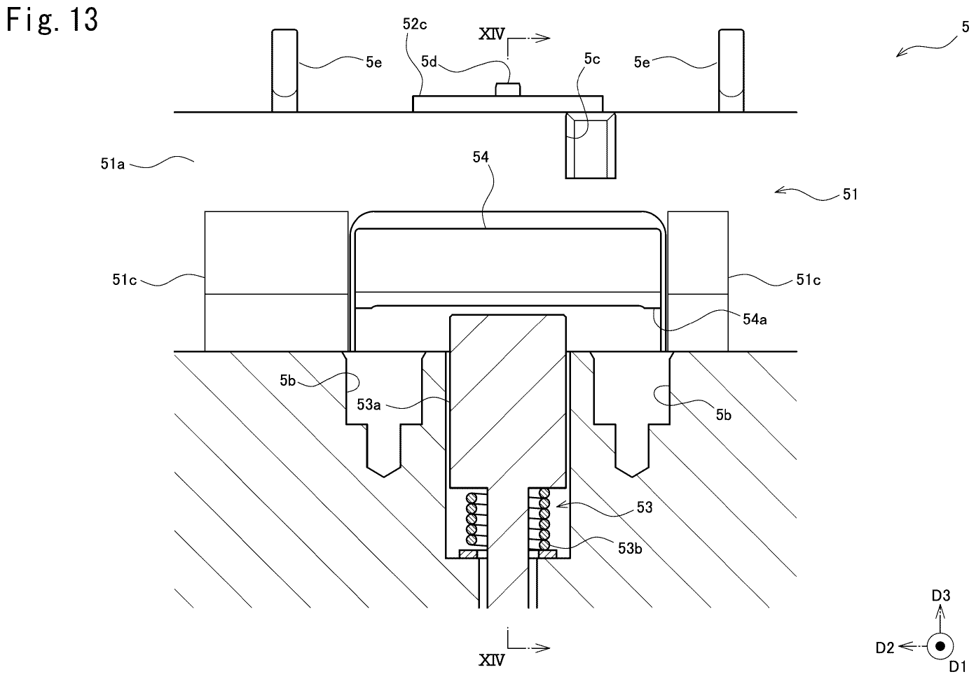

Below, embodiments of a measuring system and a measuring device are described with reference to FIG. 1 through FIG. 25. At the respective drawings, note that dimensional ratios in the drawings and actual dimensional ratios are not necessarily consistent, and note further that dimensional ratios are not necessarily consistent from drawing to drawing.

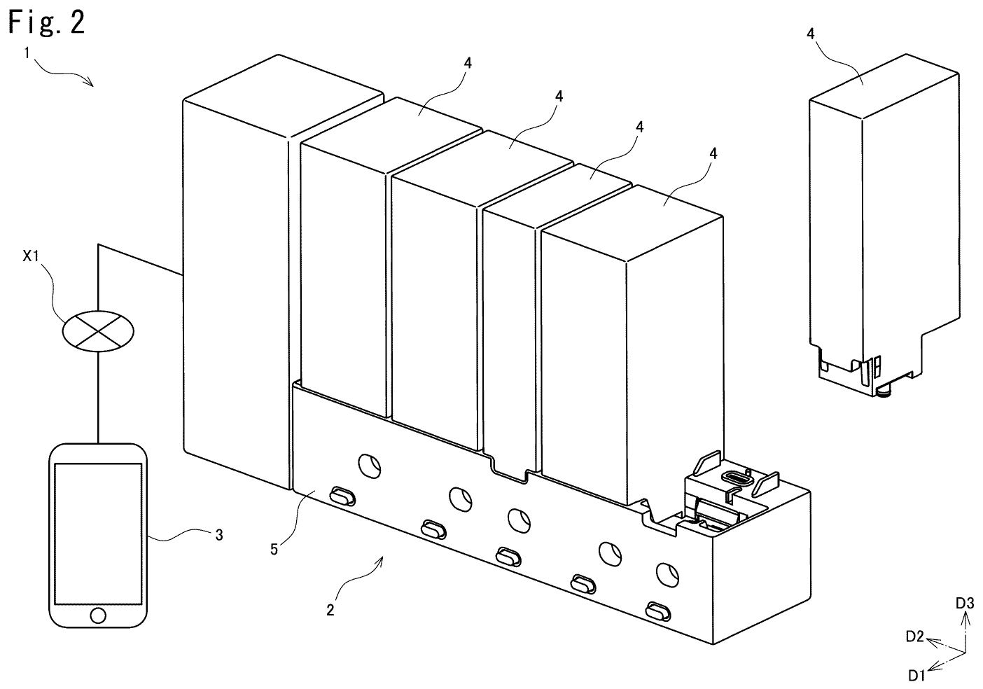

As shown in FIG. 1 and FIG. 2, measuring system 1 might, for example, comprise measuring device 2 which measures a fluid, and communication apparatus 3 which is capable of communicating with measuring device 2 by means of communication means X1. Note that there is no particular limitation with respect to the fluid, it being possible, for example, for this to include not only liquid(s), but also gas(es), mixtures of liquid(s) and gas(es), mixtures of liquid(s) and solid(s), and so forth.

While there is no particular limitation with respect thereto, communication apparatus 3 may, e.g., as is the case in the present embodiment, be a mobile terminal (e.g., a smart device, tablet computer, notebook-type personal computer, or the like). Furthermore, communication means X1 might, for example, be Wi-Fi, a wireless LAN, or other such wireless communication means; or it might, for example, be a communication cable, a wired LAN, or other such wired communication means.

Measuring device 2 might, for example, comprise a plurality (five in the present embodiment) of measurement gauges 4 which measure fluid(s), and main body 5 with respect to which the respective measurement gauges 4 are removably installed. Note that there is no particular limitation with respect to the number of measurement gauges 4 that are present, it being possible for there to be one, two to four, or six or more thereof.

So long as it is a measuring instrument that measures a fluid-related value (e.g., a characteristic value, state value, or the like), there is no particular limitation with regard to measurement gauge 4. For example, where measurement gauge 4 is a water quality meter that measures a water-related value, measurement gauge 4 might, for example, be a turbidimeter, colorimeter, pH meter, residual chlorine concentration meter, electrical conductivity meter, flow meter, water temperature meter, or the like. Note that a plurality of measurement gauges 4 may measure respectively different fluid-related values.

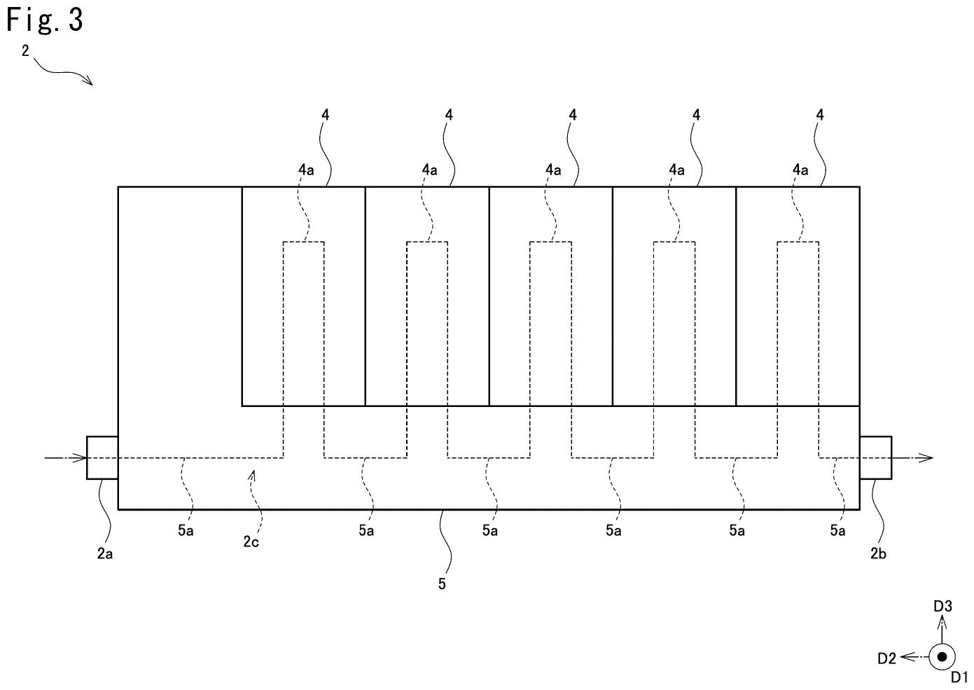

As shown in FIG. 3, measuring device 2 might, e.g., as is the case in the present embodiment, comprise fluid inlet 2a into which fluid flows, fluid outlet 2b out of which fluid flows, and fluid passage 2c through which fluid flows in passing from fluid inlet 2a to fluid outlet 2b. Moreover, although not shown in the drawings, measuring device 2 may comprise a structure in which, for example, a fluid (e.g., chemical liquid for cleaning, calibration solution for calibration, etc.) which is different from the fluid (e.g., water) serving as measurement target is made to flow through fluid passage 2c.

Furthermore, a constitution may be adopted in which, e.g., as is the case in the present embodiment, measurement gauge 4 comprises measurement fluid passage 4a, at the interior of which fluid flows, and main body 5 comprises main body fluid passage 5a, at the interior of which fluid flows, measurement fluid passage 4a and main body fluid passage 5a constituting fluid passage 2c. In addition, when measurement gauge 4 is attached to main body 5, this may cause measurement fluid passage 4a and main body fluid passage 5a to be mutually connected to constitute fluid passage 2c.

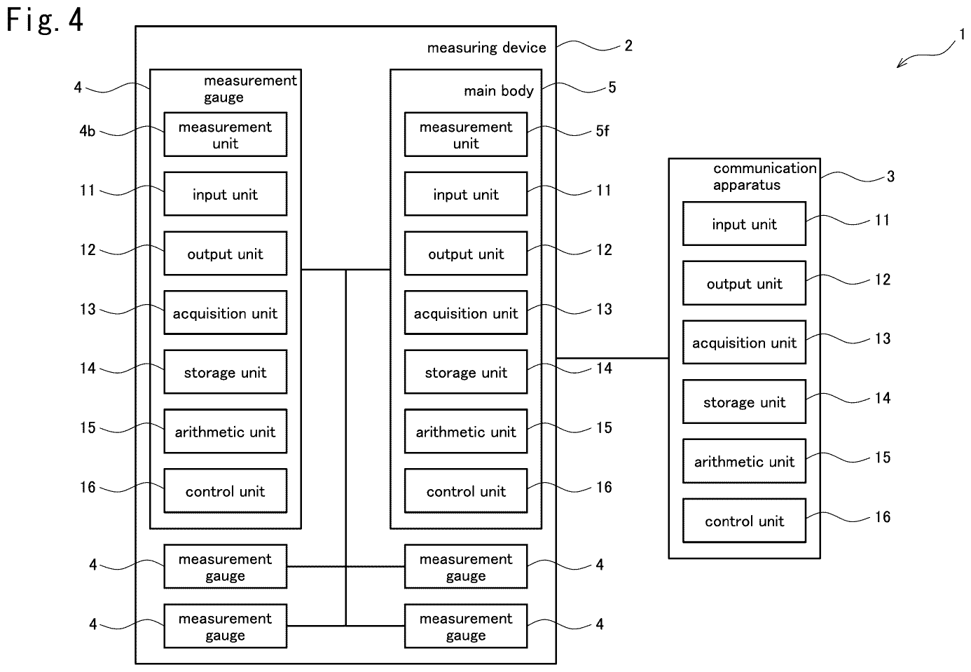

As shown in FIG. 4, measurement gauge 4 and main body 5 might, for example, respectively comprise measurement units 4b, 5f for measuring fluid(s). In addition, measuring device 2 (specifically, respective measurement gauges 4 and main body 5) and communication apparatus 3 might, e.g., as is the case in the present embodiment, comprise input unit(s) 11 at which various types of data are input, and output unit(s) 12 at which various types of data are output.

Furthermore, measuring device 2 (specifically, respective measurement gauges 4 and main body 5) and communication apparatus 3 might, e.g., as is the case in the present embodiment, comprise acquisition unit(s) 13 at which various types of data are acquired, storage unit(s) 14 at which various types of data are stored, arithmetic unit(s) 15 at which arithmetic operations are carried out on various types of data, and control unit(s) 16 at which apparatus(es) 2 (4, 5), 3 are controlled based on various types of data.

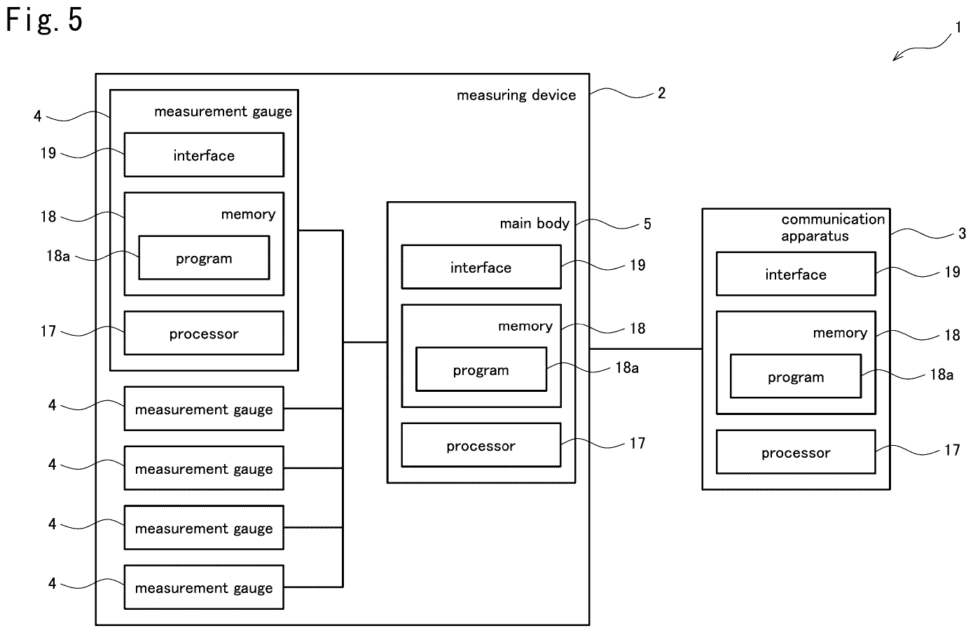

Moreover, as shown in FIG. 5, measuring device 2 (specifically, respective measurement gauges 4 and main body 5) and communication apparatus 3 may comprise computer(s) having a CPU, MPU, and/or other such processor(s) 17 (e.g., arithmetic unit 15 and control unit 16); ROM, RAM, and/or other such memory or memories 18 (e.g., acquisition unit 13 and storage unit 14); various interfaces 19 (e.g., acquisition unit 13), and so forth.

In addition, program(s) 18a stored in memory or memories 18 may be executed by processor(s) 17, and arithmetic unit(s) 15 and control unit(s) 16 of measuring device 2 (specifically, respective measurement gauges 4 and main body 5) and communication apparatus 3 may be implemented as a result of cooperation between software and hardware.

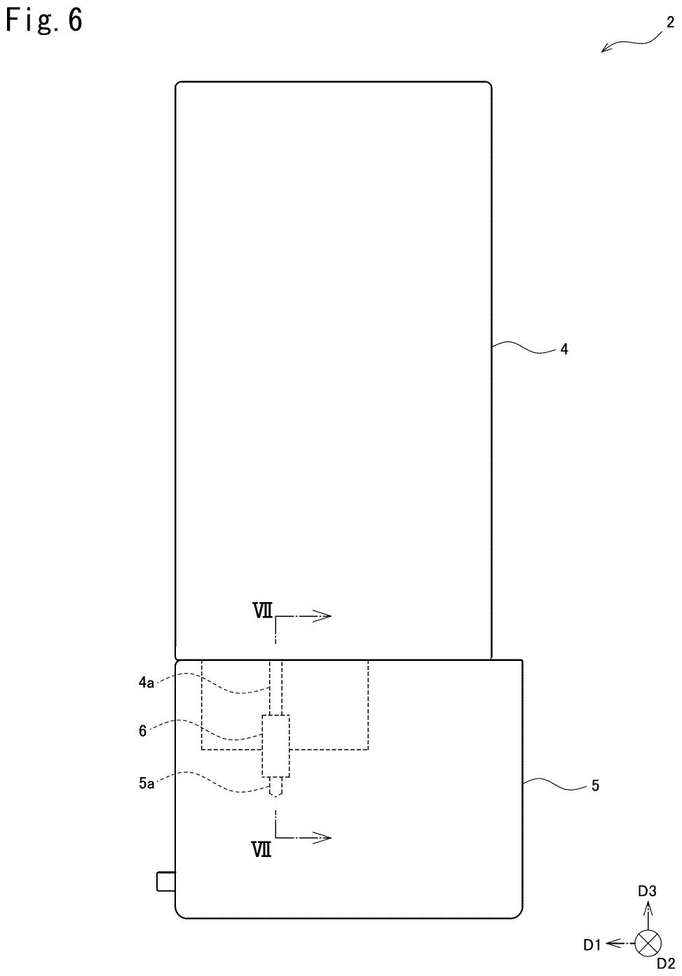

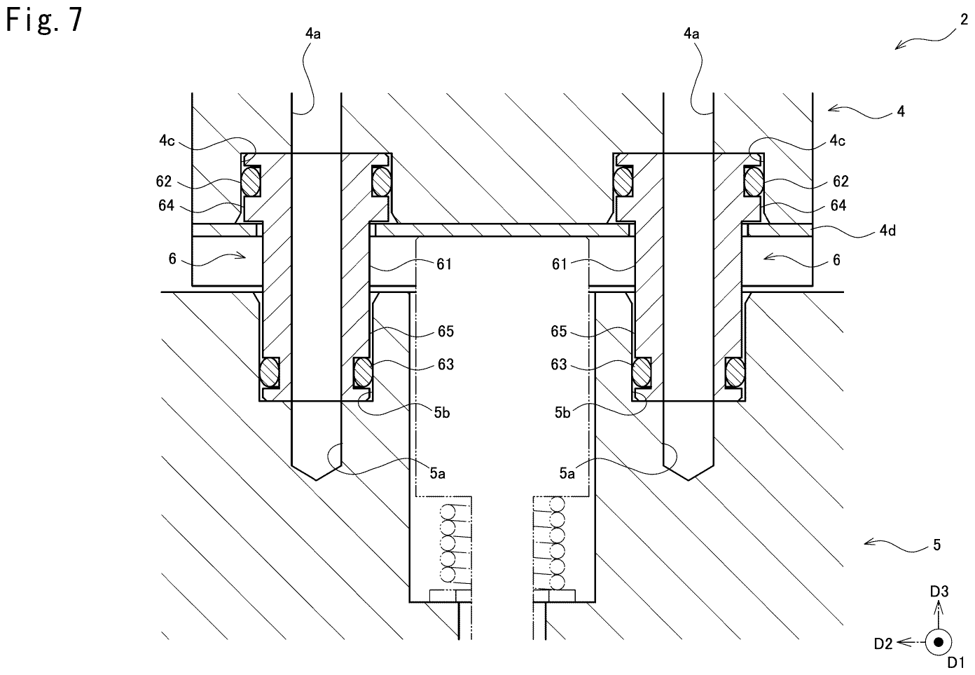

As shown in FIG. 6 and FIG. 7, measuring device 2 may, e.g., as is the case in the present embodiment, comprise connector(s) 6 which are secured to measurement gauge(s) 4 and which are for achieving structural connection between measurement gauge(s) 4 and main body 5. This will make it possible for measurement fluid passage(s) 4a of measurement gauge(s) 4 and main body fluid passage(s) 5a of main body 5 to be connected by connector(s) 6. At FIG. 7, note that a portion of main body 5 is shown in double-dash chain line.

There is no particular limitation with respect to the number of connector(s) 6 that are present, it being possible e.g., as is the case in the present embodiment, for there to be two thereof; and it is also possible for there to be one or three or more thereof. Furthermore, the two connectors 6 may, e.g., as is the case in the present embodiment, be arrayed in parallel fashion with respect to second horizontal direction D2. At the respective drawings, note that first direction D1 is first horizontal direction D1; second direction D2 is second horizontal direction D2 which is a horizontal direction that is perpendicular to first horizontal direction D1; and third direction D3 is vertical direction D3 which is perpendicular to horizontal directions D1 and D2.

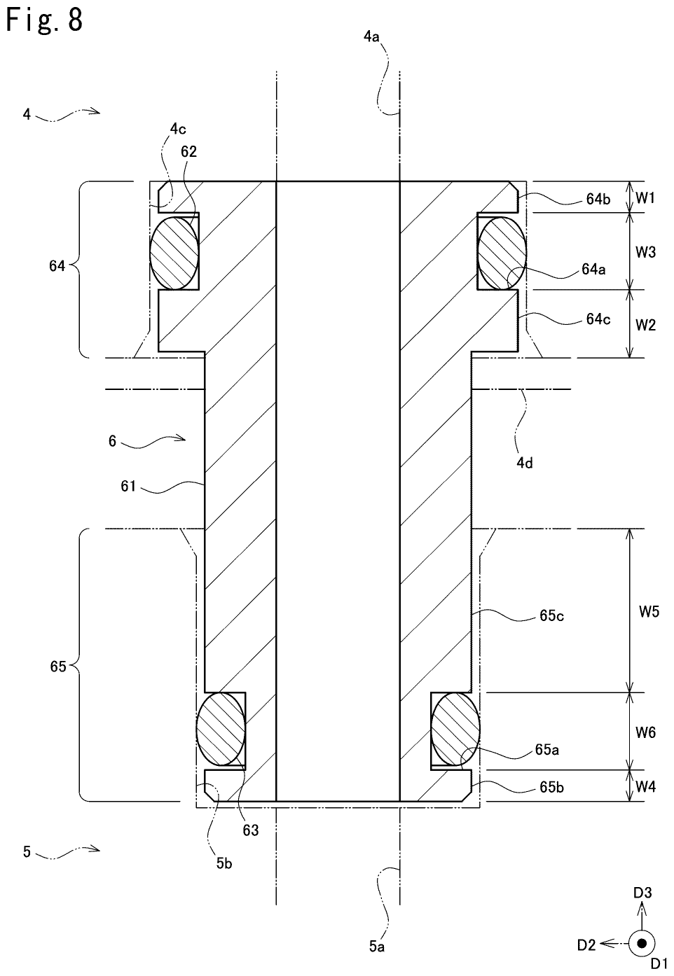

As shown in FIG. 7 and FIG. 8, connector 6 might, e.g., as is the case in the present embodiment, comprise connecting tube 61, at the interior of which fluid flows and which is a straight tube that extends linearly; first elastic body 62 which seals the region between connecting tube 61 and measurement gauge 4; and second elastic body 63 which seals the region between connecting tube 61 and main body 5. At FIG. 8, note that measurement gauge 4 and main body 5 are shown in double-dash chain line.

Measurement gauge 4 comprises recessed measurement connection port 4c which is coupled to the end of measurement fluid passage 4a; connecting tube 61 comprises, at a first end thereof, first insertion tube portion 64 which is inserted into the interior of measurement connection port 4c. In addition, first elastic body 62, which is formed to as to be annular, is arranged between the outside circumferential portion of first insertion tube portion 64 and the inside circumferential portion of measurement connection port 4c.

As a result, because first elastic body 62 will undergo elastic deformation uniformly everywhere along the entire circumference thereof, the region between the outside circumferential portion of first insertion tube portion 64 and the inside circumferential portion of measurement connection port 4c will be sealed by first elastic body 62. While there is no particular limitation with respect to the constitution of first elastic body 62, note that this might, for example, be an O-ring, it being sufficient that the constitution of first elastic body 62 be such as to permit elastic deformation in the radial direction.

Furthermore, main body 5 comprises recessed main body connection port 5b which is coupled to the end of main body fluid passage 5a; connecting tube 61 comprises, at a second end thereof, second insertion tube portion 65 which is inserted into the interior of main body connection port 5b. In addition, second elastic body 63, which is formed to as to be annular, is arranged between the outside circumferential portion of second insertion tube portion 65 and the inside circumferential portion of main body connection port 5b.

As a result, because second elastic body 63 will undergo elastic deformation uniformly everywhere along the entire circumference thereof, region between the outside the circumferential portion of second insertion tube portion 65 and the inside circumferential portion of main body connection port 5b will be sealed by second elastic body 63. While there is no particular limitation with respect to the constitution of second elastic body 63, note that this might, for example, be an O-ring, it being sufficient that the constitution of second elastic body 63 be such as to permit elastic deformation in the radial direction.

Thus, measurement fluid passage 4a is connected to main body fluid passage 5a by connecting tube 61. Moreover, because first elastic body 62 seals the region between connecting tube 61 and measurement gauge 4, and because second elastic body 63 seals the region between connecting tube 61 and main body 5, it is possible to cause measurement fluid passage 4a and main body fluid passage 5a to be definitively connected such that there is no leakage of fluid from fluid passage 2c, for example (see FIG. 3).

Note that measurement gauge 4 might, e.g., as is the case in the present embodiment, comprise retainer 4d which stops first insertion tube portion 64 from coming free of measurement connection port 4c. As a result, because connecting tube 61 is secured to measurement gauge 4, removable installation of measurement gauge 4 with respect to main body 5 will cause connector 6 to be removably installed with respect to main body 5 (e.g., see FIG. 2).

As shown in FIG. 8, first insertion tube portion 64 might, e.g., as is the case in the present embodiment, comprise first groove 64a which extends along the full extent of the outside circumference so as to contain first elastic body 62, and first flange 64b which is arranged at the tip end of first insertion tube portion 64 so as to constitute first groove 64a. Note that first insertion tube portion 64 may, e.g., as is the case in the present embodiment, be constituted from first groove 64a, first flange 64b, and first basal portion 64c which is arranged toward the center of connecting tube 61 from first groove 64a.

In addition, while there is no particular limitation with respect thereto, it is preferred that the constitution be such that, e.g., as is the case in the present embodiment, distance W1 between first groove 64a and the tip end of first insertion tube portion 64 is less than distance W2 between first groove 64a and the base end of first insertion tube portion 64. More specifically, it is preferred that the constitution be such that dimension W1 in tube axial direction (the direction of the axis of connecting tube 61) D3 of first flange 64b is less than dimension W2 in tube axial direction D3 of first basal portion 64c.

This will make it possible to reduce the size of the gap between measurement fluid passage 4a and first elastic body 62. Accordingly, because it will be possible to reduce the amount of fluid (e.g., measurement target liquid being measured, chemical liquid for cleaning, calibration solution for calibration, etc.) that enters said gap, it will be possible, for example, to suppress reduction in measurement precision.

Moreover, while there is no particular limitation with respect thereto, it is preferred that the constitution be such that, e.g., as is the case in the present embodiment, dimension W1 in tube axial direction D3 of first flange 64b is less than dimension W3 in tube axial direction D3 of first groove 64a. As a result, because it will be possible to reduce the size of the gap between measurement fluid passage 4a and first elastic body 62, this will make it possible to further reduce the amount of fluid that enters said gap.

Furthermore, second insertion tube portion 65 might, e.g., as is the case in the present embodiment, comprise second groove 65a which extends along the full extent of the outside circumference so as to contain second elastic body 63, and second flange 65b which is arranged at the tip end of second insertion tube portion 65 so as to constitute second groove 65a. Note that second insertion tube portion 65 may, e.g., as is the case in the present embodiment, be constituted from second groove 65a, second flange 65b, and second basal portion 65c which is arranged toward the center of connecting tube 61 from second groove 65a.

In addition, while there is no particular limitation with respect thereto, it is preferred that the constitution be such that, e.g., as is the case in the present embodiment, distance W4 between second groove 65a and the tip end of second insertion tube portion 65 is less than distance W5 between second groove 65a and the base end of second insertion tube portion 65. More specifically, it is preferred that the constitution be such that dimension W4 in tube axial direction D3 of second flange 65b is less than dimension W5 in tube axial direction D3 of second basal portion 65c.

This will make it possible to reduce the size of the gap between main body fluid passage 5a and second elastic body 63. Accordingly, because it will be possible to reduce the amount of fluid that enters said gap, it will be possible, for example, to suppress reduction in measurement precision.

Moreover, while there is no particular limitation with respect thereto, it is preferred that the constitution be such that, e.g., as is the case in the present embodiment, dimension W4 in tube axial direction D3 of second flange 65b is less than dimension W6 in tube axial direction D3 of second groove 65a. As a result, because it will be possible to reduce the size of the gap between main body fluid passage 5a and second elastic body 63, this will make it possible to further reduce the amount of fluid that enters said gap.

It so happens that because connector 6 is secured to measurement gauge 4, measurement gauge 4 and connector 6 are removably installable with respect to main body 5. As a result, when measurement gauge 4 and connector 6 are attached to main body 5, it is sometimes the case that connecting tube 61 (specifically, second insertion tube portion 65) will become dislocated in vertical direction D3 relative to main body connection port 5b.

While there is no particular limitation with thereto, it is therefore preferred that the constitution be such that, e.g., as is the case in the present embodiment, distance W5 between second groove 65a and the base end of second insertion tube portion 65 is greater than distance W2 between first groove 64a and the base end of first insertion tube portion 64. More specifically, it is preferred that the constitution be such that dimension W5 in tube axial direction D3 of second basal portion 65c is greater than dimension W2 in tube axial direction D3 of first basal portion 64c.

As a result, when measurement gauge 4 and connector 6 are attached to main body 5, even if second insertion tube portion 65 were to become dislocated in vertical direction (tube axial direction) D3 relative to main body connection port 5b, second elastic body 63 will definitively seal the region between the outside circumferential portion of second insertion tube portion 65 and the inside circumferential portion of main body connection port 5b. Moreover, while there is no particular limitation with respect thereto, a constitution may be adopted in which, e.g., as is the case in the present embodiment, dimension (W4+W5+W6) in tube axial direction D3 of second insertion tube portion 65 is greater than dimension (W1+W2+W3) in tube axial direction D3 of first insertion tube portion 64.

Furthermore, while there is no particular limitation with respect thereto, a constitution may be adopted in which, e.g., as is the case in the present embodiment, the maximum outside diameter of first insertion tube portion 64 is greater than the maximum outside diameter of second insertion tube portion 65. Furthermore, while there is no particular limitation with respect thereto, a constitution may be adopted in which, e.g., as is the case in the present embodiment, the outside diameter of first elastic body 62 is greater than the outside diameter of second elastic body 63.

Moreover, it is preferred, when measurement gauge 4 is attached to main body 5, that measurement connection port 4c be at a desired location relative to main body connection port 5b. Furthermore, it is preferred, after measurement gauge 4 has been attached to main body 5, that measurement connection port 4c not become displaced relative to main body connection port 5b. In accordance with the present embodiment, constitutions such as the following are therefore employed for measurement gauge 4 and main body 5. Note, however, that constitutions of measurement gauge 4 and main body 5 are not limited to the following constitutions.

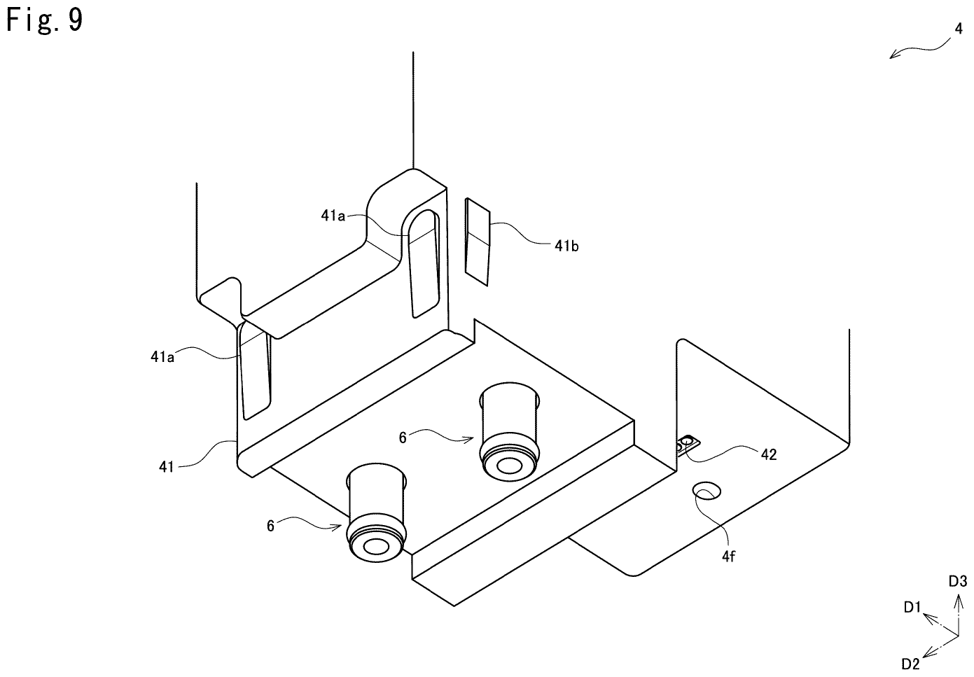

As shown in FIG. 9 through FIG. 14, a constitution may be adopted in which, e.g., as is the case in the present embodiment, main body 5 comprises a main body recess 51 which is open thereabove, and measurement gauge 4 comprises measurement insertion portion 41 at the bottom thereof which is inserted into main body recess 51. This will make it possible, when measurement insertion portion 41 is inserted into main body recess 51, for measurement gauge 4 to be attached to main body 5.

In addition, a constitution may be adopted in which, e.g., as is the case in the present embodiment, main body connection port 5b is arranged at main body recess 51, and measurement connection port 4c is arranged at measurement insertion portion 41. As a result, because connector 6 will protrude downward from measurement insertion portion 41, when measurement insertion portion 41 is pressed into main body recess 51, this will cause connector 6 to be connected to main body 5.

Main body recess 51 may, e.g., as is the case in the present embodiment, comprise a pair of first sandwiching members 51a, 51a which sandwich measurement insertion portion 41 in first horizontal direction D1, and a pair of second sandwiching members 51b, 51b which sandwich measurement insertion portion 41 in second horizontal direction D2. This will make it possible for measurement insertion portion 41 to be positioned with respect to main body recess 51 in horizontal directions D1 and D2.

Furthermore, main body 5 may, e.g., as is the case in the present embodiment, comprise a pair of third sandwiching members 5e, 5e which sandwich measurement gauge 4 in first horizontal direction D1. As a result, because measurement gauge 4 is positioned with respect to main body 5 in first horizontal direction D1, this will cause measurement insertion portion 41 to be definitively positioned with respect to main body recess 51 in first horizontal direction D1.

Moreover, measurement insertion portion 41 may, e.g., as is the case in the present embodiment, comprise first measurement protrusion 41a which protrudes in first horizontal direction D1, and second measurement protrusion 41b which protrudes in second horizontal direction D2. Furthermore, main body recess 51 may, e.g., as is the case in the present embodiment, comprise first main body protrusion(s) 51c which protrude from first sandwiching member(s) 51a in first horizontal direction D1.

Furthermore, measurement gauge 4 and main body 5 may, e.g., as is the case in the present embodiment, comprise first and second engaging members 4e, 4f, 5c, 5d which mutually engage. For example, a constitution may be adopted in which first measurement engaging member 4e is formed so as to be convex, and first main body engaging member 5c is formed so as to be concave so as to permit first measurement engaging member 4e to be inserted therewithin; and moreover, second main body engaging member 5d is formed so as to be convex, and second measurement engaging member 4f is formed so as to be concave so as to permit second main body engaging member 5d to be inserted therewithin.

Furthermore, a constitution may be adopted in which, e.g., as is the case in the present embodiment, measurement gauge 4 comprises measurement contact(s) 42 which are electrical contact(s), and main body 5 comprises contact connector portion 52 at which electrical connection is made with measurement contact(s) 42. In addition, contact connector portion 52 may comprise main body contact 52a which comes in contact with measurement contact 42, contact retaining portion 52b which retains main body contact 52a so as to permit main body contact 52a to move in vertical direction D3, and contact sealing portion 52c which possesses elasticity and which seals respective contacts 42, 52a.

Furthermore, main body 5 may, e.g., as is the case in the present embodiment, comprise lifter 53 which pushes upward on measurement gauge 4. Lifter 53 may, e.g., as is the case in the present embodiment, comprise abutting member 53a which abuts measurement gauge 4, and lifter force providing component 53b which exerts an upwardly directed elastic restoring force on abutting member 53a.

Furthermore, a constitution may be adopted in which, e.g., as is the case in the present embodiment, main body 5 comprises interlocking member 54 having interlocking portion 54a which abuts on and stops measurement insertion portion 41 from above, and measurement insertion portion 41 comprises recessed interlocked portion 41c which is abutted on and stopped by interlocking portion 54a. As a result, when interlocked portion 41c is abutted on and stopped by interlocking portion 54a, this will cause measurement insertion portion 41 to be positioned with respect to main body recess 51 in vertical direction D3, as a result of which it will be possible to inhibit measurement connection port 4c from becoming dislocated in vertical direction D3 relative to main body connection port 5b.

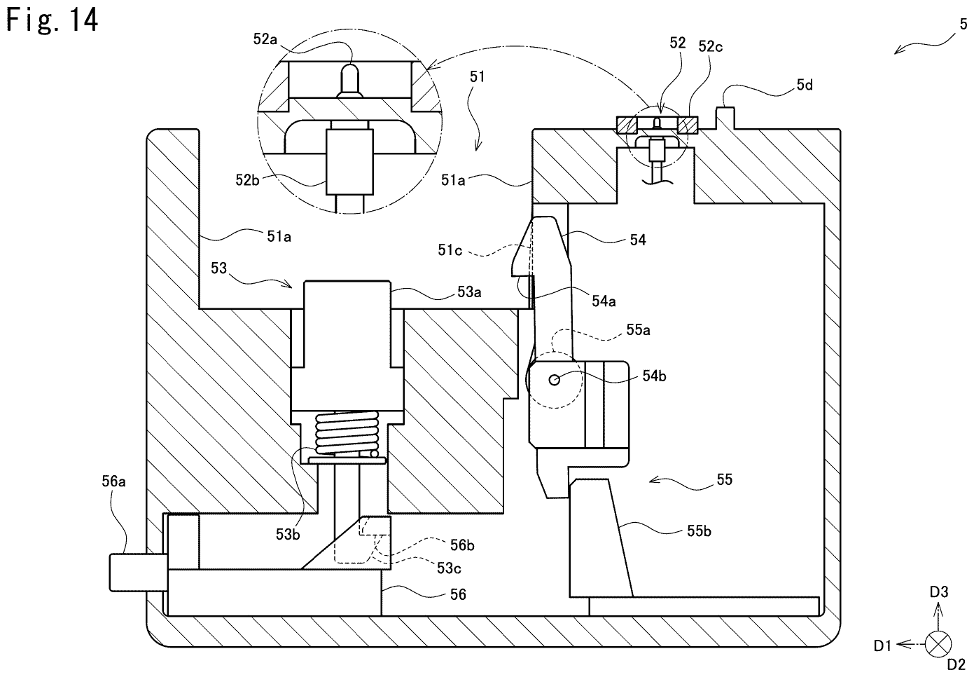

As shown in FIG. 14 through FIG. 17, main body 5 may, e.g., as is the case in the present embodiment, comprise displacing portion 55 which actuates interlocking member 54 so as to cause displacement of interlocking portion 54a. This will make it possible for interlocking portion 54a to engage in displacement between a locked position at which measurement insertion portion 41 is abutted on and stopped by displacing portion 55, and an unlocked position at which said abutment and stopping does not occur. Moreover, so as to allow it to engage in rotary displacement, interlocking member 54 might, for example, be constituted so as to be rotatable about shaft 54b which extends in second horizontal direction D2.

Displacing portion 55 may, e.g., as is the case in the present embodiment, comprise locking force providing component 55a which exerts an elastic restoring force on interlocking member 54 such as will tend to cause interlocking portion 54a to be located in the locked position; actuation member 56 which has actuation region 56a that is capable of being actuated; displacing member 55b which displaces interlocking portion 54a as a result of movement of actuation member 56; and linking mechanism 57 which connects actuation member 56 and displacing member 55b.

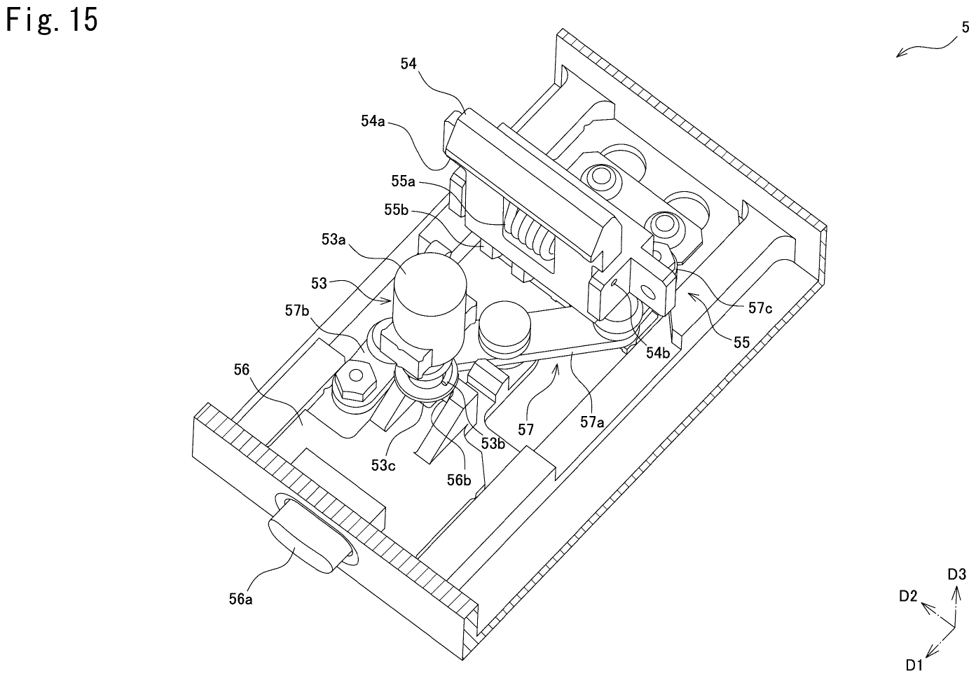

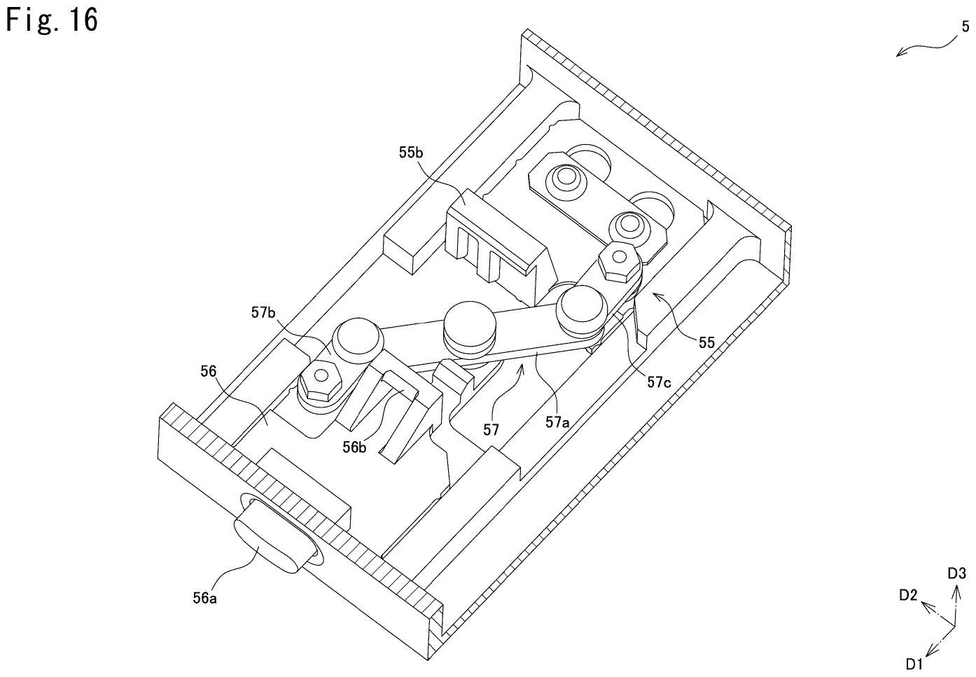

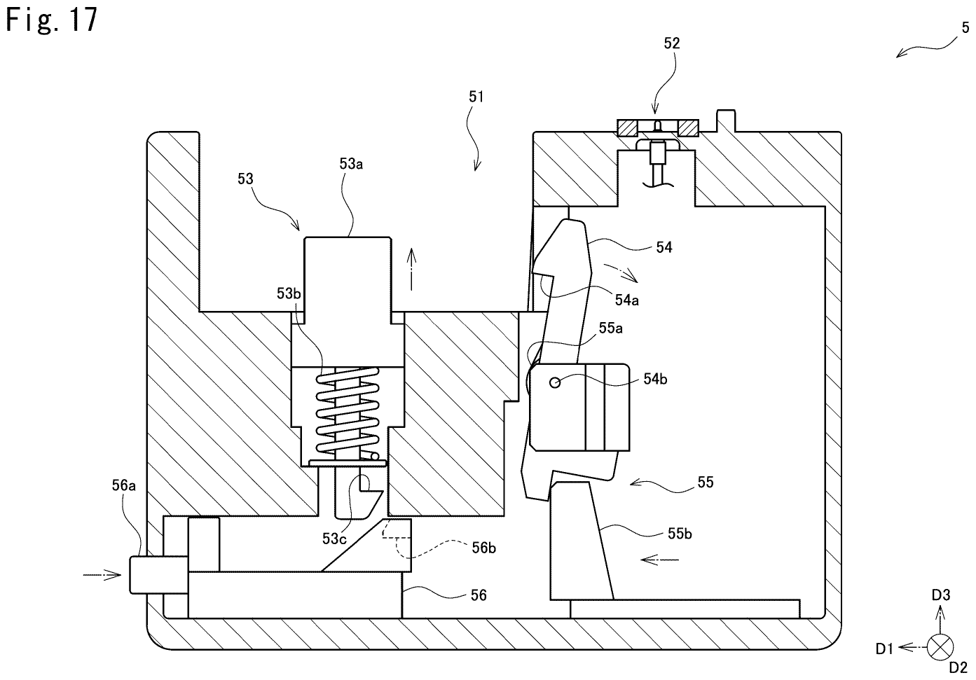

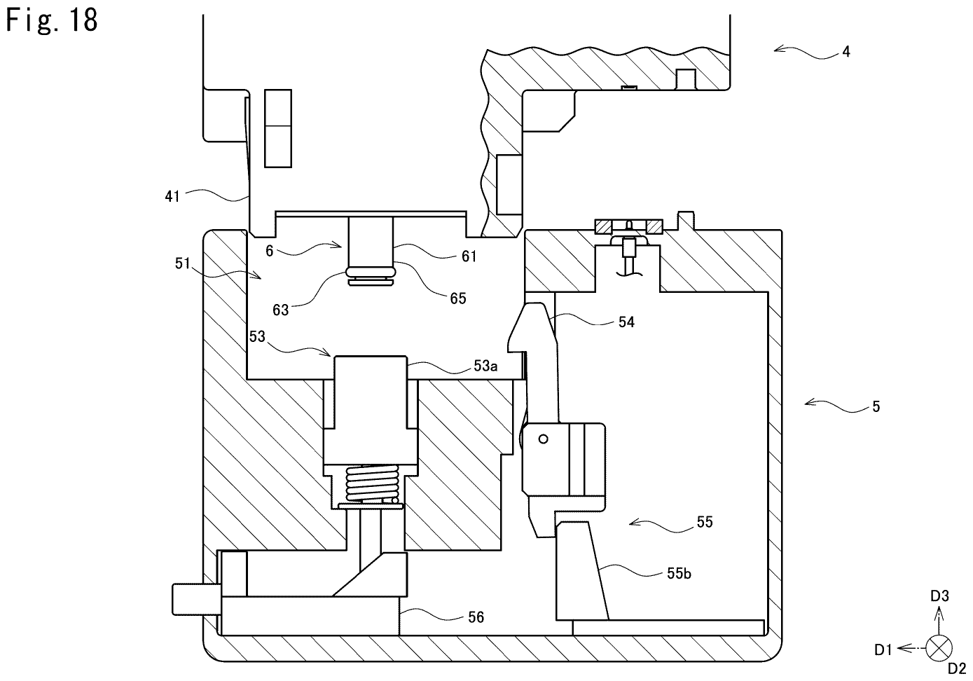

At FIG. 14 and FIG. 17 (and the same is true for FIG. 18 through FIG. 20 and FIG. 25), note that linking mechanism 57 is not shown. Furthermore, lifter 53, interlocking member 54, and displacing portion 55 are shown at FIG. 15, and a part (displacing member 55b, actuation member 56, and linking mechanism 57) of displacing portion 55 is shown at FIG. 16, but lifter 53, interlocking member 54, and the other part (locking force providing component 55a) of displacing portion 55 are not shown thereat.

For example, as shown in FIG. 14, this might be such that, when no external force acts on actuation region 56a, the elastic restoring force from locking force providing component 55a might, for example, cause interlocking portion 54a to be located at the locked position where it protrudes in first horizontal direction D1 from main body recess 51. Moreover, so as to cause abutting member 53a to be retained at standby position, abutting member 53a and actuation member 56 might comprise engaging members 53c, 56b which mutually engage.

As shown in FIG. 15 and FIG. 16, linking mechanism 57 may, e.g., as is the case in the present embodiment, comprise first link 57a which is rotatable about vertical direction D3 at a central portion thereof, second link 57b which connects actuation member 56 and first link 57a, and third link 57c which connects displacing member 55b and first link 57a. As a result, in accompaniment to movement of actuation member 56 in first horizontal direction D1, displacing member 55b will move in a direction that is opposite the direction of movement of actuation member 56.

Accordingly, e.g., as shown in FIG. 17, when actuation region 56a is pressed rightward, this will cause actuation member 56 to move rightward, in accompaniment to which displacing member 55b will move leftward. At such time, this might be such that, when displacing member 55b presses on interlocking member 54, this might, for example, cause interlocking portion 54a to be located at the unlocked position where it does not protrude in first horizontal direction D1 from main body recess 51. Furthermore, movement of actuation member 56 may cause the engagement between abutment engaging member 53c and actuation engaging member 56b to be released, such that abutting member 53a is made to move to the lifting position by lifter force providing component 53b.

A method by which measurement gauge 4 may be removably installed with respect to main body 5 will now be described with reference to FIG. 18 through FIG. 25. Note, however, that methods by which measurement gauge 4 may be removably installed with respect to main body 5 are not limited to the following method.

As shown in FIG. 18, measurement insertion portion 41 is inserted into main body recess 51. At such time, it is possible, for example, for this to be such that abutting member 53a is made to be retained in the standby position in advance as a result of causing abutting member 53a to be pressed downward and of causing abutting member 53a and actuation member 56 to come into engagement. Moreover, it is also possible, for example, for this to be such that abutting member 53a is made to move to the standby position as a result of causing measurement insertion portion 41 to depress abutting member 53a as it is inserted into main body recess 51.

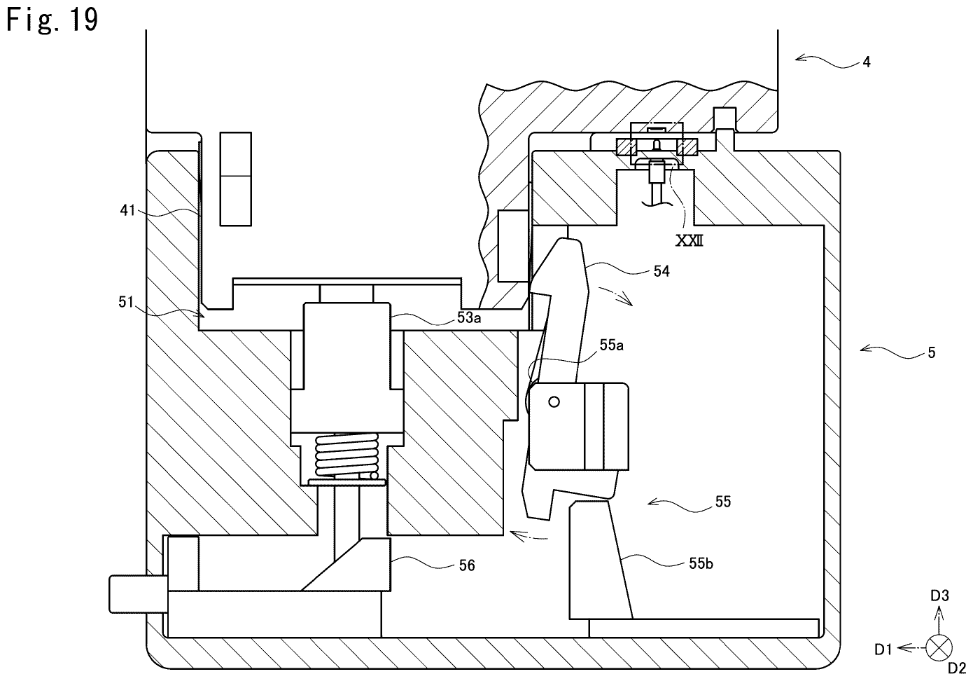

As shown in FIG. 19, because when measurement insertion portion 41 is pressed further into main body recess 51, this causes measurement insertion portion 41 to abut interlocking member 54, interlocking member 54 overcomes the elastic restoring force of locking force providing component 55a and rotates. At such time, because interlocking member 54 rotates so as to move away from displacing member 55b, displacing member 55b does not move. As a result, because actuation member 56 does not move, it is possible for the engagement between abutting member 53a and actuation member 56 to be definitively maintained.

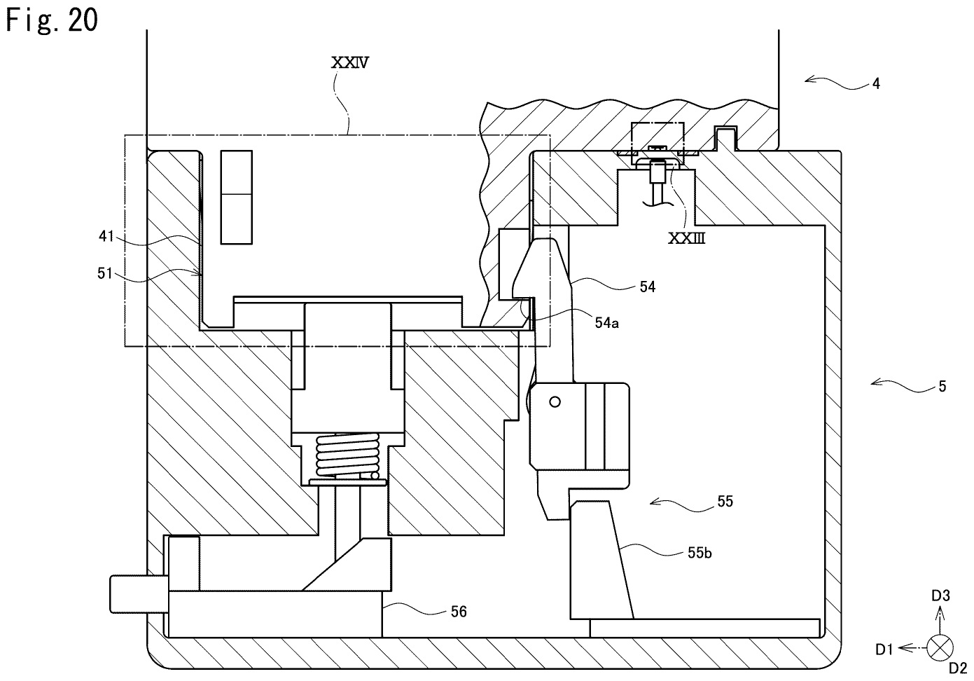

As shown in FIG. 20, when measurement insertion portion 41 is pressed further into main body recess 51, this causes interlocking portion 54a to be located at the locked position. As a result, because interlocking portion 54a abuts on and stops measurement insertion portion 41 from above, this causes measurement insertion portion 41 to be positioned with respect to main body recess 51 in vertical direction D3.

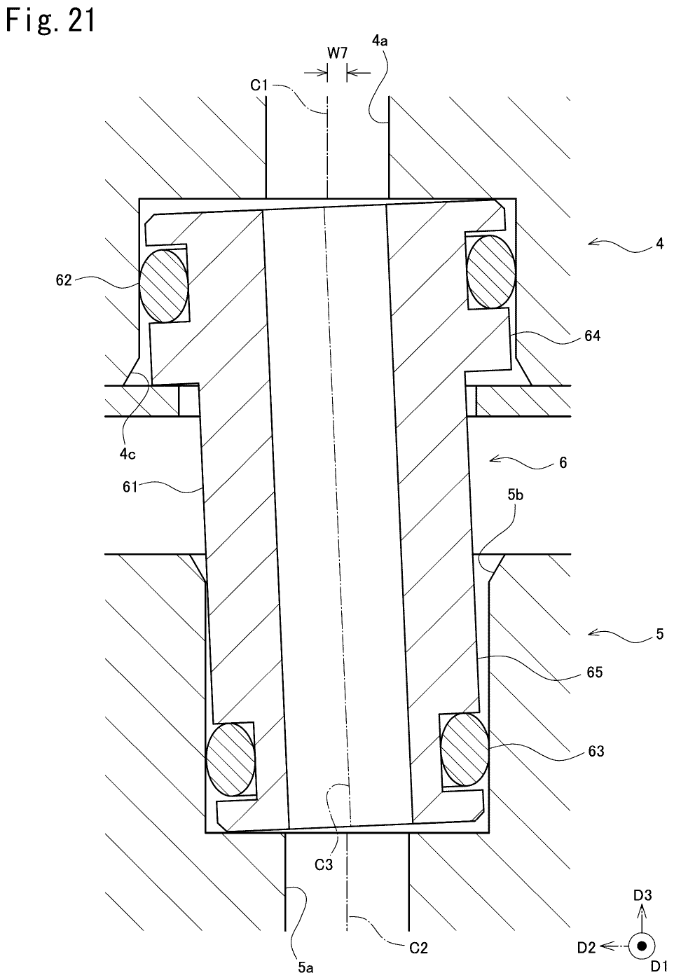

At such time, as shown in FIG. 21, existence, for example, of an attachment error, manufacturing error, or the like may cause the location of measurement fluid passage 4a with respect to main body fluid passage 5a to be misaligned relative to the reference location therefor. For example, suppose that central axis C1 of measurement fluid passage 4a (measurement connection port 4c) is misaligned by a prescribed distance W7 in second horizontal direction D2 relative to central axis C2 of main body fluid passage 5a (main body connection port 5b).

This being the case, first elastic body 62 will undergo elastic deformation uniformly everywhere along the entire circumference in the region between the outside circumferential portion of first insertion tube portion 64 and the inside circumferential portion of measurement connection port 4c, and second elastic body 63 will undergo elastic deformation uniformly everywhere along the entire circumference in the region between the outside circumferential portion of second insertion tube portion 65 and the inside circumferential portion of main body connection port 5b. This will cause central axis C3 of connecting tube 61 to be inclined relative to respective central axes C1, C2.

In addition, the region between the outside circumferential portion of first insertion tube portion 64 and the inside circumferential portion of measurement connection port 4c will be sealed by first elastic body 62, and the region between the outside circumferential portion of second insertion tube portion 65 and the inside circumferential portion of main body connection port 5b will be sealed by second elastic body 63. Accordingly, even if the location of measurement fluid passage 4a with respect to main body fluid passage 5a were to become misaligned relative to the reference location therefor, connector 6 will be capable of causing measurement fluid passage 4a and main body fluid passage 5a to be definitively connected.

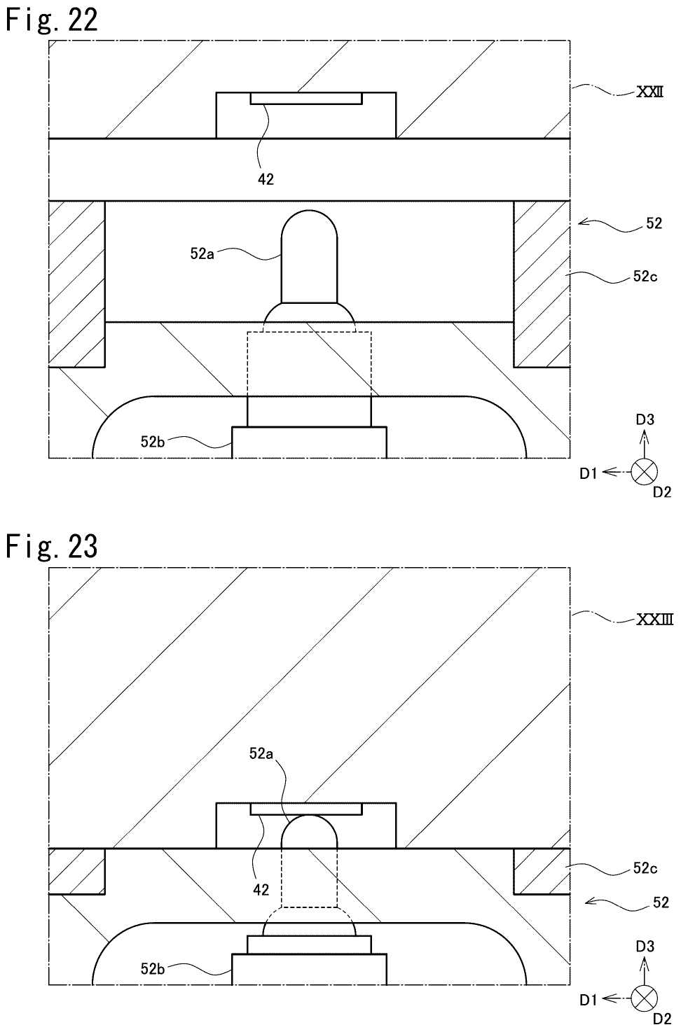

Furthermore, when interlocking portion 54a abuts on and stops measurement insertion portion 41 from above and measurement gauge 4 is attached to main body 5 (see FIG. 20), this causes main body contact 52a to go from the state in FIG. 22 to the state in FIG. 23. More specifically, as shown in FIG. 23, when main body contact 52a is pressed on by measurement contact 42, this causes main body contact 52a to move downward relative to contact retaining portion 52b.

Thus, when interlocking portion 54a abuts on and stops measurement insertion portion 41 from above and measurement gauge 4 is attached to main body 5, not only does this make it possible to cause measurement fluid passage 4a and main body fluid passage 5a to be connected to constitute fluid passage 2c (see FIG. 3) but it also makes it possible to cause measurement contact 42 and main body contact 52a to be electrically connected. Note that measurement contact 42 and main body contact 52a may, e.g., as is the case in the present embodiment, be arranged above connector 6.

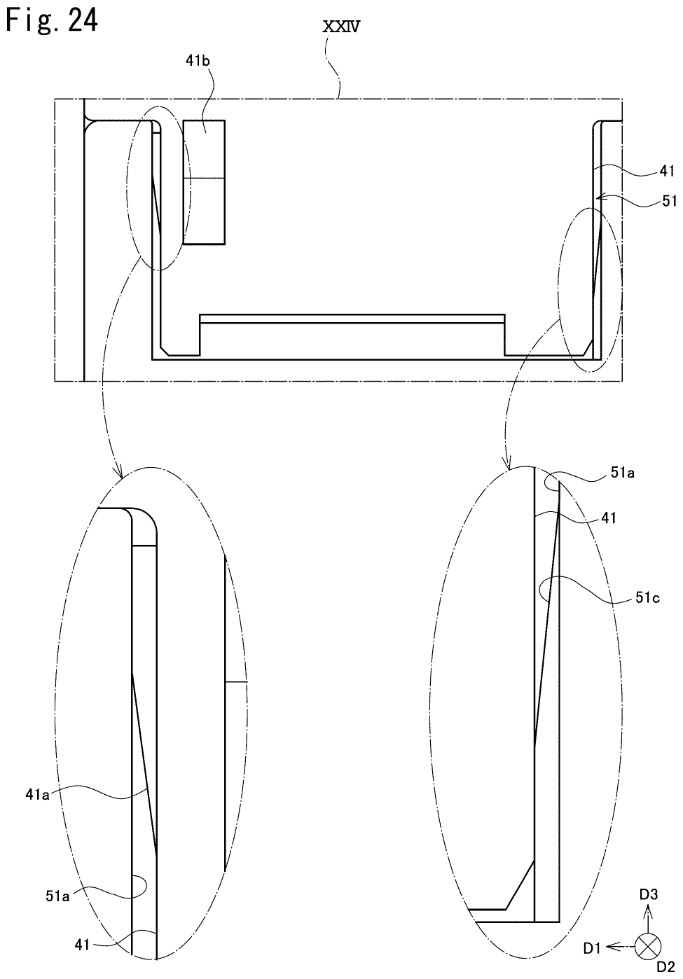

Furthermore, when interlocking portion 54a abuts on and stops measurement insertion portion 41 from above and measurement gauge 4 is attached to main body 5 (see FIG. 20), this causes first measurement protrusion 41a to reduce the size of the gap between measurement insertion portion 41 and main body recess 51, and also causes first main body protrusion 51c to reduce the size of the gap between main body recess 51 and measurement insertion portion 41, as shown in FIG. 24. This makes it possible to inhibit measurement insertion portion 41 from becoming dislocated in first horizontal direction D1 relative to main body recess 51.

Moreover, a constitution may be adopted in which, e.g., as is the case in the present embodiment, the top end of first measurement protrusion 41a is arranged above the center in vertical direction D3 of measurement insertion portion 41, and the bottom end of first main body protrusion 51c is arranged below the center in vertical direction D3 of main body recess 51. This will make it possible for first measurement protrusion 41a to reduce the size of the gap between the upper portion of main body recess 51 and the upper portion of measurement insertion portion 41, and will make it possible for first main body protrusion 51c to reduce the size of the gap between the lower portion of main body recess 51 and the lower portion of measurement insertion portion 41.

Accordingly, it is possible as a result of cooperation between first measurement protrusion 41a and first main body protrusion 51c to effectively inhibit measurement insertion portion 41 from becoming dislocated in first horizontal direction D1 relative to main body recess 51. Note that the bottom end of first measurement protrusion 41a may, e.g., as is the case in the present embodiment, be arranged below the top end of first main body protrusion 51c.

In addition, it is possible, e.g., as is the case in the present embodiment, to adopt a constitution in which the height with which first measurement protrusion 41a protrudes increases as one proceeds upward, and the height with which first main body protrusion 51c protrudes increases as one proceeds downward. This will facilitate insertion of measurement insertion portion 41 into main body recess 51 despite presence of first measurement protrusion 41a and first main body protrusion 51c.

Moreover, a constitution may be adopted in which, e.g., as is the case in the present embodiment, the top end of second measurement protrusion 41b is arranged above the center in vertical direction D3 of measurement insertion portion 41, and the height with which second measurement protrusion 41b protrudes increases as one proceeds upward. This will facilitate insertion of measurement insertion portion 41 into main body recess 51 despite presence of second measurement protrusion 41b.

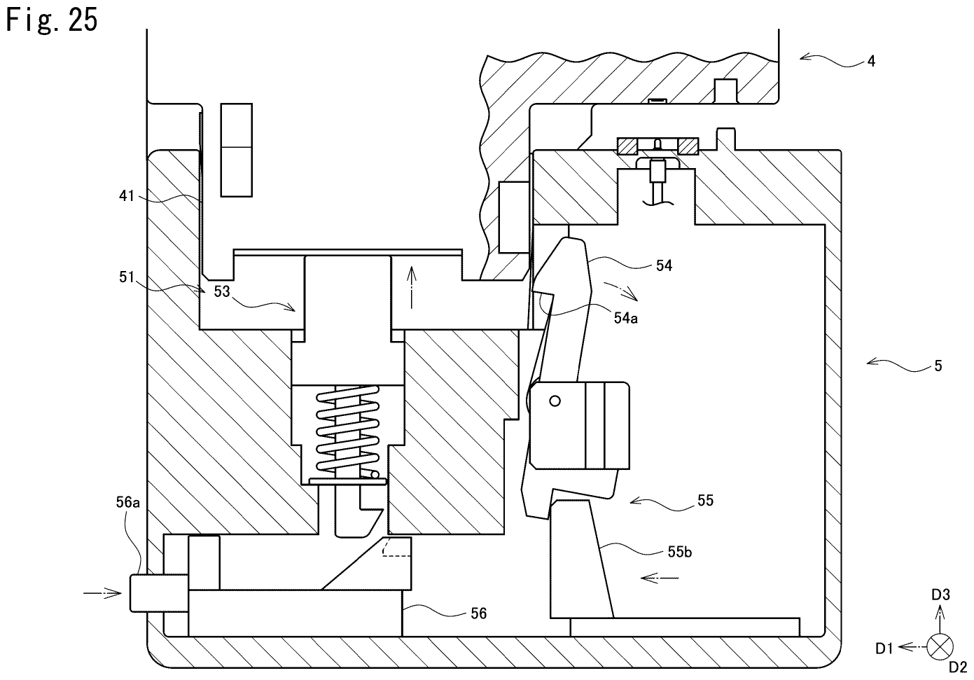

Thereafter, as shown in FIG. 25, when actuation region 56a is pressed on, this causes interlocking portion 54a to be displaced to the unlocked position, and causes lifter 53 to lift measurement gauge 4 upward. At this time, because measurement gauge 4 moves in tube axial direction D3, second insertion tube portion 65 will move in tube axial direction D3 relative to main body connection port 5b. As a result, this will make it possible, for example, to suppress occurrence of a situation in which imbalanced force(s) might otherwise act on second elastic body 63 and connecting tube 61 when detaching measurement gauge 4 and connector 6 from main body 5.

Furthermore, frictional forces, adhesive forces, or other such forces might, for example, arise in the region between second elastic body 63 and main body connection port 5b. Where this is the case, lifting of measurement gauge 4 by lifter 53 may, for example, cause second elastic body 63 to move to a location at which it is able to come free of main body connection port 5b. This will facilitate detachment of measurement gauge 4 and connector 6 from main body 5.

As described above, as in the present embodiment, the measuring system 1 comprises:
the measuring device 2; and
a communication apparatus 3 that is capable of communicating with the measuring device 2.

And, the measuring device 2 comprises:
a measurement gauge 4 which measures a fluid;
a main body 5 with respect to which the measurement gauge 4 is removably installable; and
a connector 6 which connects the measurement gauge 4 and the main body 5;
wherein the measurement gauge 4 comprises
   a measurement fluid passage 4a through which the fluid flows, and
   a recessed measurement connection port 4c which is coupled to an end of the measurement fluid passage 4a;
wherein the main body 5 comprises
   a main body fluid passage 5a through which the fluid flows, and
   a recessed main body connection port 5b which is coupled to an end of the main body fluid passage 5a; and
wherein the connector 6 comprises
   a connecting tube 61, a first end of which is inserted into an interior of the measurement connection port 4c, and a second end of which is inserted into an interior of the main body connection port 5b;
   an annular first elastic body 62 which seals a region between an outside circumferential portion of the first end of the connecting tube 61 and an inside circumferential portion of the measurement connection port 4c; and
   an annular second elastic body 63 which seals a region between an outside circumferential portion of the second end of the connecting tube 61 and an inside circumferential portion of the main body connection port 5b.

In accordance with such constitution, a first end of connecting tube 61 is inserted into the interior of measurement connection port 4c, and a second end of connecting tube 61 is inserted into the interior of main body connection port 5b. In addition, because annular first elastic body 62 is arranged between the outside circumferential portion at the first end of connecting tube 61 and the inside circumferential portion of measurement connection port 4c, first elastic body 62 will undergo elastic deformation uniformly everywhere along the entire circumference thereof. This will make it possible for the region between the outside circumferential portion at the first end of connecting tube 61 and the inside circumferential portion of measurement connection port 4c to be sealed by first elastic body 62.

Furthermore, because annular second elastic body 63 is arranged between the outside circumferential portion at the second end of connecting tube 61 and the inside circumferential portion of main body connection port 5b, second elastic body 63 will undergo elastic deformation uniformly everywhere along the entire circumference thereof. This will make it possible for the region between the outside circumferential portion at the second end of connecting tube 61 and the inside circumferential portion of main body connection port 5b to be sealed by second elastic body 63. Accordingly, connector 6 will be capable of causing measurement fluid passage 4a and main body fluid passage 5a to be definitively connected.

Further, as in the present embodiment, it is preferred that the measuring device 2 includes a configuration in which:
the connecting tube 61 comprises, at the first end thereof, a first insertion tube portion 64 which is inserted into the interior of the measurement connection port 4c;
the first insertion tube portion 64 comprises a first groove 64a which extends along a full extent of an outside circumference thereof so as to contain the first elastic body 62; and
a distance W1 between the first groove 64a and a tip end of the first insertion tube portion 64 is less than a distance W2 between the first groove 64a and a base end of the first insertion tube portion 64.

In accordance with such constitution, first elastic body 62 is contained within first groove 64a which extends along the full extent of the outside circumference of first insertion tube portion 64. In addition, because distance W1 between first groove 64a and the tip end of first insertion tube portion 64 is less than distance W2 between first groove 64a and the base end of first insertion tube portion 64, it is possible to reduce the size of the gap between measurement fluid passage 4a and first elastic body 62.

Further, as in the present embodiment, it is preferred that the measuring device 2 includes a configuration in which:
the connecting tube 61 comprises, at the second end thereof, a second insertion tube portion 65 which is inserted into the interior of the main body connection port 5b;
the second insertion tube portion 65 comprises a second groove 65a which extends along a full extent of an outside circumference thereof so as to contain the second elastic body 63; and
a distance W4 between the second groove 65a and a tip end of the second insertion tube portion 65 is less than a distance W5 between the second groove 65a and a base end of the second insertion tube portion 65.

In accordance with such constitution, second elastic body 63 is contained within second groove 65a which extends along the full extent of the outside circumference of second insertion tube portion 65. In addition, because distance W4 between second groove 65a and the tip end of second insertion tube portion 65 is less than distance W5 between second groove 65a and the base end of second insertion tube portion 65, it is possible to reduce the size of the gap between main body fluid passage 5a and second elastic body 63.

Further, as in the present embodiment, it is preferred that the measuring device 2 includes a configuration in which:
the measurement gauge 4 comprises a retainer 4d which is for securing the connector 6 and which stops the first end of the connecting tube 61 from coming free of the measurement connection port 4c.

In accordance with such constitution, because a first end of connecting tube 61 is stopped by retainer 4d from coming free of measurement connection port 4c, connector 6 is secured to measurement gauge 4. This makes it possible for removable installation of measurement gauge 4 with respect to main body 5 to cause connector 6 to be removably installed with respect to main body 5.

Further, as in the present embodiment, it is preferred that the measuring device 2 includes a configuration in which:
the connecting tube 61 comprises
   a first insertion tube portion 64 which is arranged at the first end of the connecting tube 61 and which is inserted into the interior of the measurement connection port 4c, and
   a second insertion tube portion 65 which is arranged at the second end of the connecting tube 61 and which is inserted into the interior of the main body connection port 5b;
the first insertion tube portion 64 comprises a first groove 64a which extends along a full extent of an outside circumference thereof so as to contain the first elastic body 62;
the second insertion tube portion 65 comprises a second groove 65a which extends along a full extent of an outside circumference thereof so as to contain the second elastic body 63; and
a distance W5 between the second groove 65a and a base end of the second insertion tube portion 65 is greater than a distance W2 between the first groove 64a and a base end of the first insertion tube portion 64.

In accordance with such constitution, because distance W5 between second groove 65a and the base end of second insertion tube portion 65 is greater than distance W2 between first groove 64a and the base end of first insertion tube portion 64, the distance between second elastic body 63 and the base end of second insertion tube portion 65 is large. As a result, when measurement gauge 4 and connector 6 are attached to main body 5, even if second insertion tube portion 65 were to become dislocated relative to main body connection port 5b in tube axial direction D3, second elastic body 63 will definitively seal the region between the outside circumferential portion of second insertion tube portion 65 and the inside circumferential portion of main body connection port 5b.

Further, as in the present embodiment, it is preferred that the measuring device 2 includes a configuration in which:
the main body 5 further comprises a main body recess 51 which is open thereabove;
the measurement gauge 4 further comprises a measurement insertion portion 41 which is inserted into the main body recess 51;
the main body connection port 5b is arranged at the main body recess 51;
the measurement connection port 4c is arranged at the measurement insertion portion 41; and
the main body recess 51 comprises
   a pair of first sandwiching members 51a, 51a which sandwich the measurement insertion portion 41 in a first horizontal direction D1, and
   a pair of second sandwiching members 51b, 51b which sandwich the measurement insertion portion 41 in a second horizontal direction D2 that is perpendicular to the first horizontal direction D1.

In accordance with such constitution, a pair of first sandwiching members 51a, 51a sandwich measurement insertion portion 41 in first horizontal direction D1, and a pair of second sandwiching members 51b, 51b sandwich measurement insertion portion 41 in second horizontal direction D2. This makes it possible for measurement insertion portion 41 to be positioned with respect to main body recess 51 in horizontal directions D1 and D2. In addition, because main body connection port 5b is arranged at main body recess 51, and measurement connection port 4c is arranged at measurement insertion portion 41, it will be possible to inhibit measurement connection port 4c from becoming dislocated in horizontal directions D1 and D2 relative to main body connection port 5b.

Further, as in the present embodiment, it is preferred that the measuring device 2 includes a configuration in which:
the main body 5 further comprises
   an interlocking portion 54a that abuts on and stops the measurement insertion portion 41 from above, and
   a displacing portion 55 that causes the interlocking portion 54a to be displaced between a locked position at which the measurement insertion portion 41 is abutted on and stopped by the interlocking portion 54a, and an unlocked position at which said abutment and stopping does not occur.

In accordance with such constitution, because interlocking portion 54a, when in the locked position, abuts on and stops measurement insertion portion 41 from above, this causes measurement insertion portion 41 to be positioned with respect to main body recess 51 in vertical direction D3. This makes it possible to inhibit measurement connection port 4c from becoming dislocated in vertical direction D3 relative to main body connection port 5b.

Furthermore, when displacing portion 55 causes interlocking portion 54a to be displaced to the unlocked position, this causes the abutment and stopping by interlocking portion 54a to be released. This makes it possible for measurement gauge 4 and connector 6 to be detached from main body 5.

Further, as in the present embodiment, it is preferred that the measuring device 2 includes a configuration in which:
the main body recess 51 comprises a main body protrusion (first main body protrusion in the present embodiment) 51c which protrudes in the first horizontal direction D1 from the first sandwiching member 51a;
a bottom end of the main body protrusion 51c is arranged below a center in a vertical direction D3 of the main body recess 51; and
a height with which the main body protrusion 51c protrudes increases as one proceeds downward.

In accordance with such constitution, because main body protrusion 51c protrudes in first horizontal direction D1 from first sandwiching member 51a, main body protrusion 51c reduces the size of the gap between main body recess 51 and measurement insertion portion 41. This makes it possible to inhibit measurement insertion portion 41 from becoming dislocated in first horizontal direction D1 relative to main body recess 51.

Furthermore, the bottom end of main body protrusion 51c is arranged below the center in vertical direction D3 of main body recess 51; and moreover, the height with which main body protrusion 51c protrudes increases as one proceeds downward. This will facilitate insertion of measurement insertion portion 41 into main body recess 51 despite presence of main body protrusion 51c.

Further, as in the present embodiment, it is preferred that the measuring device 2 includes a configuration in which:
the measurement insertion portion 41 comprises a measurement protrusion (first measurement protrusion in the present embodiment) 41a which protrudes in the first horizontal direction D1;
a top end of the measurement protrusion 41a is arranged above a center in a vertical direction D3 of the measurement insertion portion 41; and
a height with which the measurement protrusion 41a protrudes increases as one proceeds upward.

In accordance with such constitution, because measurement protrusion 41a protrudes in first horizontal direction D1, measurement protrusion 41a reduces the size of the gap between measurement insertion portion 41 and main body recess 51. This makes it possible to inhibit measurement insertion portion 41 from becoming dislocated in first horizontal direction D1 relative to main body recess 51.

Furthermore, the top end of measurement protrusion 41a is arranged above the center in vertical direction D3 of measurement insertion portion 41; and moreover, the height with which measurement protrusion 41a protrudes increases as one proceeds upward. This will facilitate insertion of measurement insertion portion 41 into main body recess 51 despite presence of measurement protrusion 41a.

Further, as in the present embodiment, it is preferred that the measuring device 2 includes a configuration in which:
the main body 5 further comprises a lifter 53 which lifts the measurement gauge 4 in a tube axial direction D3 of the connecting tube 61, in a direction such as will move it away from the main body 5.

In accordance with such constitution, because lifter 53 lifts measurement gauge 4 in tube axial direction D3, in a direction such as will move it away from main body 5, measurement gauge 4 is made to move away from main body 5 in tube axial direction D3. As a result, second insertion tube portion 65 is made to move in tube axial direction D3 relative to main body connection port 5b.

Further, as in the present embodiment, it is preferred that the measuring device 2 includes a configuration in which:
the lifter 53 lifts the measurement gauge 4 to a location at which the second elastic body 63 is able to come free of the main body connection port 5b.

In accordance with such constitution, lifting of measurement gauge 4 by lifter 53 causes second elastic body 63 to move to a location at which it can be made to come free of main body connection port 5b. This will facilitate detachment of measurement gauge 4 and connector 6 from main body 5.

Further, as in the present embodiment, it is preferred that the measuring device 2 includes a configuration in which:
the first insertion tube portion 64 further comprises a first flange 64b which is arranged at a tip end of the first insertion tube portion 64 so as to constitute the first groove 64a; and
a dimension W1 in a tube axial direction D3 of the first flange 64b is less than a dimension W3 in the tube axial direction D3 of the first groove 64a.

In accordance with such constitution, because first flange 64b is arranged at the tip end of first insertion tube portion 64, first groove 64a is constituted by first flange 64b. In addition, because dimension W1 in tube axial direction D3 of first flange 64b is less than dimension W3 in tube axial direction D3 of first groove 64a, it is possible to further reduce the size of the gap between measurement fluid passage 4a and first elastic body 62.

Further, as in the present embodiment, it is preferred that the measuring device 2 includes a configuration in which:
the second insertion tube portion 65 further comprises a second flange 65b which is arranged at a tip end of the second insertion tube portion 65 so as to constitute the second groove 65a; and
a dimension W4 in the tube axial direction D3 of the second flange 65b is less than a dimension W6 in the tube axial direction D3 of the second groove 65a.

In accordance with such constitution, because second flange 65b is arranged at the tip end of second insertion tube portion 65, second groove 65a is constituted by second flange 65b. In addition, because dimension W4 in tube axial direction D3 of second flange 65b is less than dimension W6 in tube axial direction D3 of second groove 65a, it is possible to further reduce the size of the gap between main body fluid passage 5a and second elastic body 63.

The measuring system 1 and the measuring device 2 are not limited to the configuration of the embodiment described above, and the effects are not limited to those described above. It goes without saying that the measuring system 1 and the measuring device 2 can be variously modified without departing from the scope of the subject matter of the present invention. For example, the constituents, methods, and the like of various modified examples described below may be arbitrarily selected and employed as the constituents, methods, and the like of the embodiments described above, as a matter of course.

(1) The constitution of measuring device 2 associated with the foregoing embodiment is such that connector 6 is secured to measurement gauge 4, and is removably installable with respect to main body 5. However, measuring device 2 is not limited to such constitution. For example, it is also possible to adopt a constitution in which connector 6 is secured to main body 5, and is removably installable with respect to measurement gauge 4. Furthermore, for example, it is also possible to adopt a constitution in which connector 6 is respectively removably installable with respect to measurement gauge 4 and main body 5.

(2) Furthermore, the constitution of measuring device 2 associated with the foregoing embodiment is such that first insertion tube portion 64 comprises first groove 64a for containing first elastic body 62, and second insertion tube portion 65 comprises second groove 65a for containing second elastic body 63. However, measuring device 2 is not limited to such constitution. For example, it is also possible to adopt a constitution in which first insertion tube portion 64 is of the same diameter everywhere along the entire length thereof in tube axial direction D3. Furthermore, for example, it is also possible to adopt a constitution in which second insertion tube portion 65 is of the same diameter everywhere along the entire length thereof in tube axial direction D3.

(3) Furthermore, the constitution of measuring device 2 associated with the foregoing embodiment is such that main body 5 comprises a main body recess 51 which is open thereabove, and measurement gauge 4 comprises measurement insertion portion 41 which is inserted into main body recess 51, insertion of measurement insertion portion 41 into main body recess 51 causing measurement gauge 4 to be attached to main body 5. However, measuring device 2 is not limited to such constitution.

For example, it is also possible to adopt a constitution in which measurement gauge 4 comprises a bottom surface that is planar, and main body 5 comprises a top surface that is planar, measurement gauge 4 being attached to main body 5 as a result of causing measurement gauge 4 to be placed on main body 5 so as to cause the bottom surface of measurement gauge 4 to come in contact with the top surface of main body 5.

(4) Furthermore, at measuring device 2, main body recess 51 may, for example, further comprise a second main body protrusion which protrudes in second horizontal direction D2 from second sandwiching member 51b. In addition, it is also possible, for example, to adopt a constitution in which the bottom end of the second main body protrusion is arranged below the center in vertical direction D3 of main body recess 51, and the height with which the second main body protrusion protrudes increases as one proceeds downward.

This will make it possible for second measurement protrusion 41b to reduce the size of the gap between the upper portion of main body recess 51 and the upper portion of measurement insertion portion 41, and will make it possible for the second main body protrusion to reduce the size of the gap between the lower portion of main body recess 51 and the lower portion of measurement insertion portion 41. Accordingly, it will be possible as a result of cooperation between second measurement protrusion 41b and the second main body protrusion to effectively inhibit measurement insertion portion 41 from becoming dislocated in second horizontal direction D2 relative to main body recess 51.

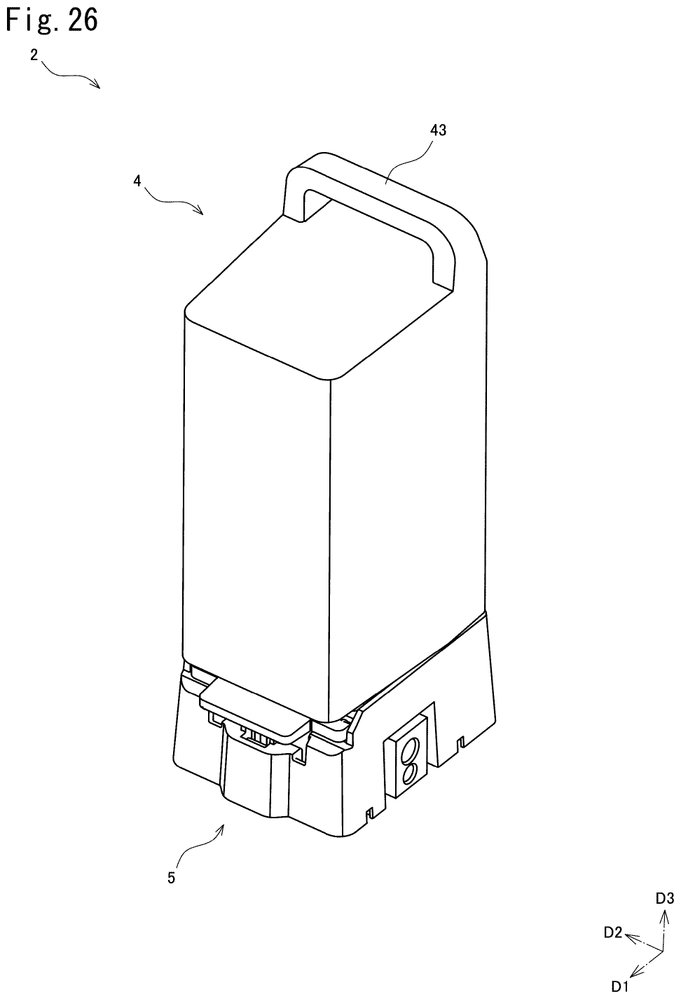

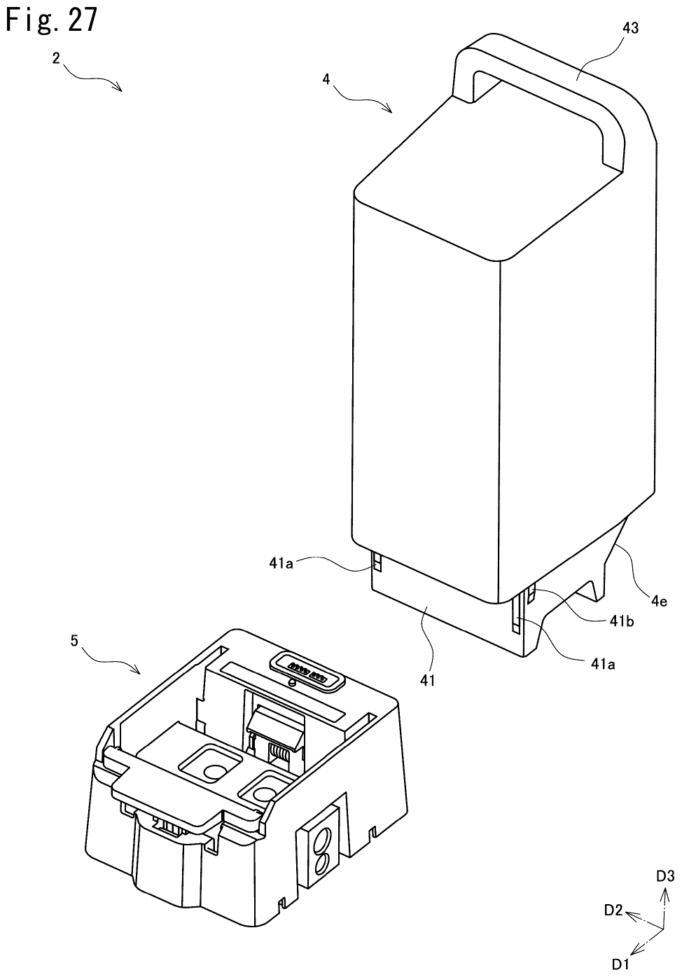

(5) Furthermore, the constitution of measuring device 2 associated with the foregoing embodiment is such that removably installable with respect to main body 5 are a plurality of measurement gauges 4. However, measuring device 2 is not limited to such constitution. For example, as shown in FIG. 26 through FIG. 31, it is also possible to adopt a constitution in which one measurement gauge 4 is removably installable with respect to main body 5. In addition, at measuring device 2 associated with FIG. 26 through FIG. 31, a plurality of main bodies 5 may be mutually connected to constitute a one fluid passage 2c (not shown in FIG. 26 through FIG. 31).

The constitution of measuring device 2 associated with FIG. 26 through FIG. 31 will be described with reference to FIG. 26 through FIG. 29.

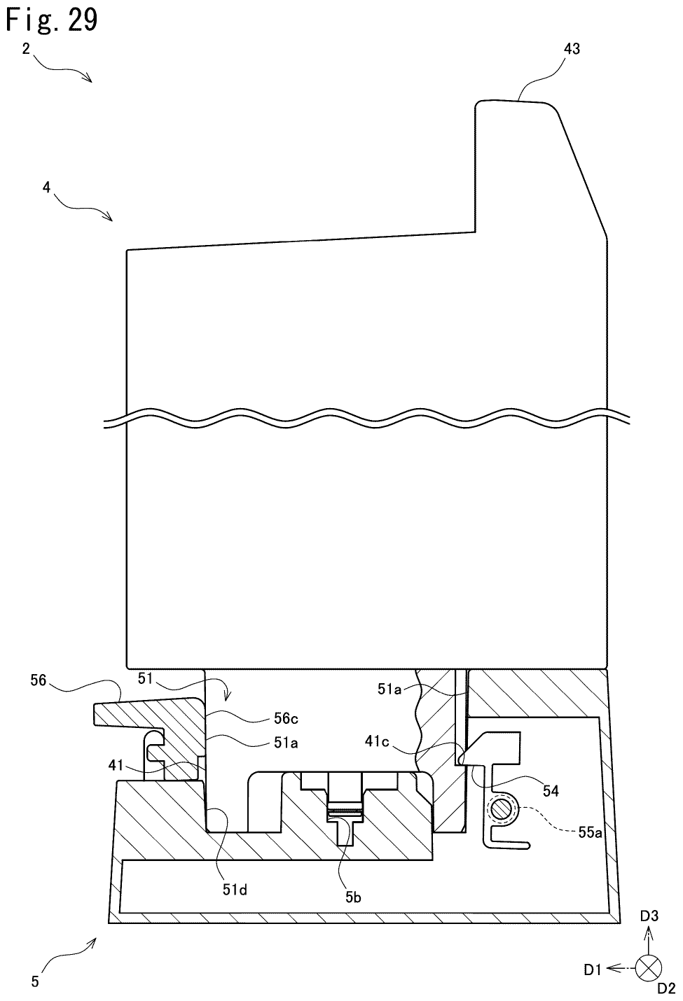

As shown in FIG. 26 through FIG. 31, measurement gauge 4 comprises grip region 43, which is capable of being gripped, in a region which is at the top end and end at a first side (the side opposite the direction of the arrow in first horizontal direction D1) in first horizontal direction D1. Moreover, interlocked portion 41c (see FIG. 29 through FIG. 31) of measurement insertion portion 41 is arranged at the end at the first side in first horizontal direction D1 of measurement insertion portion 41.

Main body 5 comprises interlocking member 54 which abuts on and stops interlocked portion 41c of measurement insertion portion 41 from above, locking force providing member 55a which exerts an elastic restoring force on interlocking member 54, and actuation member 56 which is capable of being actuated. Main body recess 51 comprises a pair of first sandwiching members 51a, 51a that sandwich measurement insertion portion 41 in first horizontal direction D1, and a pair of second sandwiching members 51b, 51b that sandwich measurement insertion portion 41 in second horizontal direction D2 which is perpendicular to first horizontal direction D1.

Interlocking portion 54 is rotatably connected to main body recess 51. As a result, interlocking member 54 engages in displacement between a locked position at which interlocked portion 41c of measurement insertion portion 41 is abutted on and stopped from above, and an unlocked position at which said abutment and stopping does not occur. In addition, locking force providing member 55a exerts an elastic restoring force on interlocking member 54 such as will tend to cause interlocking member 54 to be located in the locked position. While there is no particular limitation with respect thereto, locking force providing member 55a might, for example, be a spring.

First sandwiching member 51a which is arranged at a second side (the side in the direction of the arrow in first horizontal direction D1) in first horizontal direction D1 comprises lower sandwiching portion 51d that opposes a region which is at a lower portion and end at the second side in first horizontal direction D1 of measurement insertion portion 41. In addition, first sandwiching member 51a which is arranged at the second side in first horizontal direction D1 is constituted from lower sandwiching portion 51d and actuation member 56. Moreover. lower sandwiching portion 51d might, for example, abut measurement insertion portion 41, and might, for example, be separated from measurement insertion portion 41 such that there is a gap therebetween.

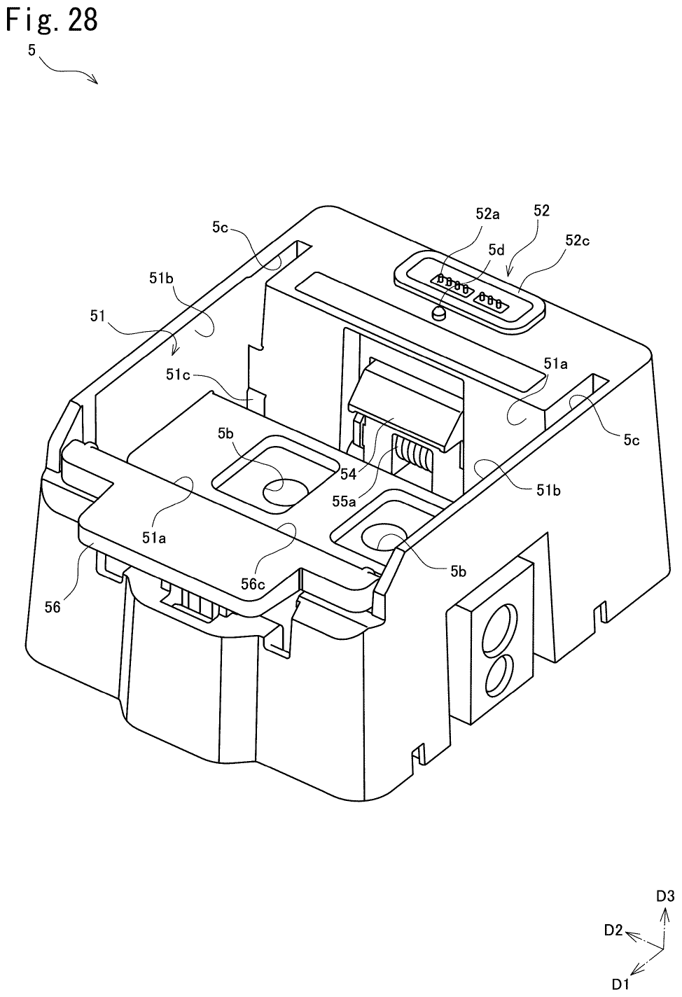

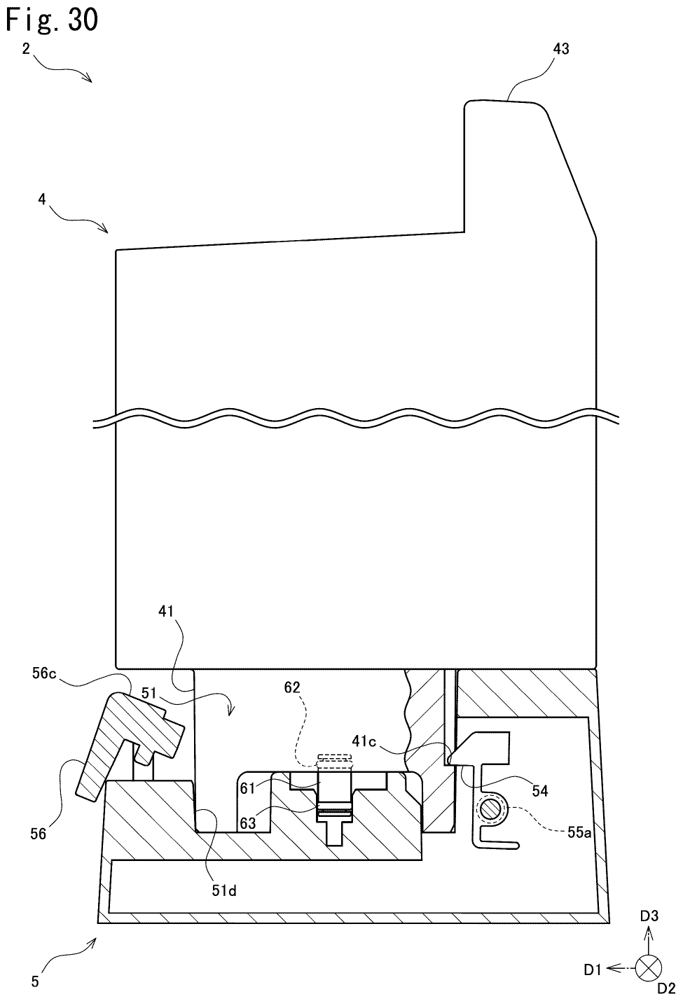

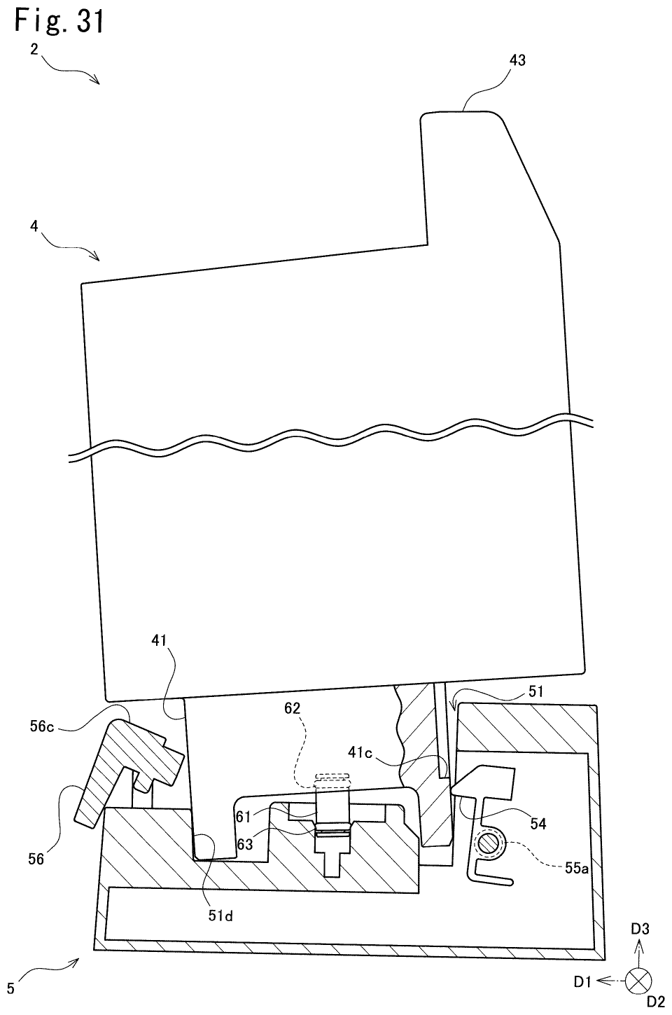

Actuation member 56 is capable of being actuated so as to engage in displacement relative to lower sandwiching portion 51d between a sandwiched position (see FIG. 28 and FIG. 29) at which it abuts the end at the second side in first horizontal direction D1 of measurement insertion portion 41, and a nonsandwiched position (see FIG. 30 and FIG. 31) at which it is separated from measurement insertion portion 41. Moreover, actuation member 56 comprises upper sandwiching portion 56c which, when located in the sandwiched position, abuts the end at the second side in first horizontal direction D1 of measurement insertion portion 41 above lower sandwiching portion 51d.

Next, at measuring device 2 associated with FIG. 26 through FIG. 31, a method by which measurement gauge 4 may be removably installed with respect to main body 5 will be described with reference to FIG. 29 through FIG. 31. Note, however, that methods by which measurement gauge 4 may be removably installed with respect to main body 5 are not limited to the following method.

First, as shown in FIG. 29, when actuation member 56 is located in the sandwiched position, this causes measurement insertion portion 41 to be sandwiched between a pair of first sandwiching members 51a, 51a. In addition, interlocking member 54, when in the locked position, abuts on and stops interlocked portion 41c of measurement insertion portion 41 from above; and moreover, because locking force providing member 55a exerts an elastic restoring force on interlocking member 54, measurement gauge 4 is attached to main body 5. As a result, measurement gauge 4 does not become dislodged from main body 5.

In addition, when measurement gauge 4 is being detached from main body 5, actuation of actuation member 56 causes actuation member 56 to be located in the nonsandwiched position as shown in FIG. 30. As a result, actuation member 56 will be separated from measurement insertion portion 41. In addition, as shown in FIG. 31, grip region 43 is gripped, and measurement gauge 4 is made to rotate (rotate in a direction opposite the clockwise direction at FIG. 31) about lower sandwiching portion 51d as pivot point. As a result, because measurement insertion portion 41 comes free of main body recess 51, measurement gauge 4 can be detached from main body unit 5.

At this time, because grip region 43 is arranged at a location distant from lower sandwiching portion 51d, the amount of force needed to overcome the elastic restoring force of locking force providing member 55a and cause measurement gauge 4 to rotate is small. Furthermore, when measurement gauge 4 is made to rotate about lower sandwiching portion 51d as pivot point, first elastic body 62 and second elastic body 63 undergo elastic deformation. Because this causes connecting tube 61 to be displaced relative to measurement insertion portion 41, detachment of measurement gauge 4 from main body 5 is facilitated.

Conversely, when measurement gauge 4 is being attached to main body 5, grip region 43 is gripped, as shown in FIG. 31, and measurement gauge 4 is made to rotate (rotate in the clockwise direction at FIG. 31) about lower sandwiching portion 51d as pivot point. As a result, as shown in FIG. 30, measurement insertion portion 41 will be inserted into the interior of main body recess 51.

In addition, as shown in FIG. 29, actuation of actuation member 56 causes actuation member 56 to be located in the sandwiched position. As a result, when actuation member 56 abuts measurement insertion portion 41, this causes measurement insertion portion 41 to be sandwiched between the pair of first sandwiching members 51a, 51a, as a result of which measurement gauge 4 is attached to main body 5.

Thu, as shown in FIG. 26 through FIG. 31, it is preferred that the measuring device 20 includes a configuration in which:
the main body 5 further comprises a main body recess 51 which is open thereabove;
the measurement gauge 4 further comprises a measurement insertion portion 41 which is inserted into the main body recess 51;
the main body connection port 5b is arranged at the main body recess 51;
the measurement connection port 4c (not shown in FIG. 26 through FIG. 31) is arranged at the measurement insertion portion 41; and
the main body recess 51 comprises
   a pair of first sandwiching members 51a, 51a which sandwich the measurement insertion portion 41 in a first horizontal direction D1, and
   a pair of second sandwiching members 51b, 51b which sandwich the measurement insertion portion 41 in a second horizontal direction D2 that is perpendicular to the first horizontal direction D1.

Further, as shown in FIG. 26 through FIG. 31, it is preferred that the measuring device 20 includes a configuration in which:
the measurement gauge 4 further comprises a grip region 43 which is capable of being gripped and which is arranged at end at a first side in a first horizontal direction D1 thereof and a top end thereof;
the main body 5 comprises
   an interlocking member 54 that is displaced between a locked position at which an end at a first side in the first horizontal direction D1 of the measurement insertion portion 41 is abutted on and stopped from above, and an unlocked position at which said abutment and stopping does not occur; and
   a locking force providing member 55a which exerts an elastic restoring force on the interlocking member 54 so as to cause the interlocking member 54 to be located in the locked position; and
that first sandwiching member 51a which of the pair of first sandwiching members 51a, 51a is arranged at a second side in the first horizontal direction D1 comprises
   a lower sandwiching portion 51d which opposes a lower portion and end at a second side in the first horizontal direction D1 of the measurement insertion portion 41; and
   an actuation member 56 which when actuated, is capable of displacing between a sandwiched position at which it abuts an end at a second side in the first horizontal direction D1 of the measurement insertion portion 41 above the lower sandwiching portion 51d, and a nonsandwiched position at which it is separated from the measurement insertion portion 41.

In accordance with such constitution, when actuation member 56 is located in the sandwiched position, this causes measurement insertion portion 41 to be sandwiched between the pair of first sandwiching members 51a, 51a. In addition, interlocking member 54, when in the locked position, abuts on and stops the end at the first side in first horizontal direction D1 of measurement insertion portion 41 from above; and moreover, because locking force providing member 55a exerts an elastic restoring force on interlocking member 54, measurement gauge 4 is attached to main body 5.

On the other hand, when actuation member 56 is located in the nonsandwiched position, grip region 43 is gripped, and measurement gauge 4 is made to rotate about lower sandwiching portion 51d as pivot point, as a result of which measurement insertion portion 41 can be made to go in or out of main body recess 51. At this time, because grip region 43 is arranged at a location distant from lower sandwiching portion 51d, the amount of force needed to overcome the elastic restoring force of locking force providing member 55a and cause measurement gauge 4 to rotate is small.

Furthermore, when measurement gauge 4 is made to rotate about lower sandwiching portion 51d as pivot point, first elastic body 62 and second elastic body 63 undergo elastic deformation. Because this causes connecting tube 61 to be displaced relative to measurement connection port 4c of measurement insertion portion 41, removable installation of measurement gauge 4 with respect to main body 5 is facilitated.

### REFERENCE CHARACTERS LIST

1 measuring system
2 measuring device
2a fluid inlet
2b fluid outlet
2c fluid passage
3 communication apparatus
4 measurement gauge
4a fluid passage
4b measurement unit
4c measurement connection port
4d retainer
4e first measurement engaging member
4f second measurement engaging member
5 main body
5a main body fluid passage
5b main body connection port
5c first main body engaging member
5d second main body engaging member
5e third sandwiching member
5f measurement unit
6 connector
11 input unit
12 output unit
13 acquisition unit
14 storage unit
15 arithmetic unit
16 control unit
17 processor
18 memory
18a program
19 interface
41 measurement insertion portion
41a first measurement protrusion
41b second measurement protrusion
41c interlocked portion
42 measurement contact
43 grip region
51 main body recess
51a first sandwiching member
51b second sandwiching member
51c first main body protrusion
51d lower sandwiching portion
52 contact connector portion
52a main body contact
52b contact retaining portion
52c contact sealing portion
53 lifter
53a abutting member
53b lifter force providing component
53c abutment engaging member
54 interlocking member
54a interlocking portion
54b shaft
55 displacing portion
55a locking force providing component
55b displacing member
56 actuation member
56a actuation region
56b actuation engaging member
56c upper sandwiching portion
57 linking mechanism
57a first link
57b second link
57c third link
61 connecting tube
62 first elastic body
63 second elastic body
64 first insertion tube portion
64a first groove
64b first flange
64c first basal portion
65 second insertion tube portion
65a second groove
65b second flange
65c second basal portion
D1 first horizontal direction
D2 second horizontal direction
D3 vertical direction
X1 communication means

1. A measuring device comprising:
a measurement gauge which measures a fluid;
a main body with respect to which the measurement gauge is removably installable; and
a connector which connects the measurement gauge and the main body;
wherein the measurement gauge comprises
a measurement fluid passage through which the fluid flows, and
a recessed measurement connection port which is coupled to an end of the measurement fluid passage;
wherein the main body comprises
a main body fluid passage through which the fluid flows, and
a recessed main body connection port which is coupled to an end of the main body fluid passage; and
wherein the connector comprises
a connecting tube, a first end of which is inserted into an interior of the measurement connection port, and a second end of which is inserted into an interior of the main body connection port;
an annular first elastic body which seals a region between an outside circumferential portion of the first end of the connecting tube and an inside circumferential portion of the measurement connection port; and
an annular second elastic body which seals a region between an outside circumferential portion of the second end of the connecting tube and an inside circumferential portion of the main body connection port.

2. The measuring device according to claim 1 wherein
the connecting tube comprises, at the first end of the connecting tube, a first insertion tube portion which is inserted into the interior of the measurement connection port;
the first insertion tube portion comprises a first groove which extends along a full extent of an outside circumference of the first insertion tube portion so as to contain the first elastic body; and
a distance between the first groove and a tip end of the first insertion tube portion is less than a distance between the first groove and a base end of the first insertion tube portion.

3. The measuring device according to claim 1 wherein
the connecting tube comprises, at the second end of the connecting tube, a second insertion tube portion which is inserted into the interior of the main body connection port;
the second insertion tube portion comprises a second groove which extends along a full extent of an outside circumference of the second insertion tube portion so as to contain the second elastic body; and
a distance between the second groove and a tip end of the second insertion tube portion is less than a distance between the second groove and a base end of the second insertion tube portion.

4. The measuring device according to claim 1 wherein the measurement gauge, comprises a retainer which is for securing the connector and which stops the first end of the connecting tube from coming free of the measurement connection port.

5. The measuring device according to claim 4 wherein
the connecting tube comprises
a first insertion tube portion which is arranged at the first end of the connecting tube and which is inserted into the interior of the measurement connection port, and
a second insertion tube portion which is arranged at the second end of the connecting tube and which is inserted into the interior of the main body connection port;
the first insertion tube portion comprises a first groove which extends along a full extent of an outside circumference of the first insertion tube portion so as to contain the first elastic body;
the second insertion tube portion comprises a second groove which extends along a full extent of an outside circumference of the second insertion tube portion so as to contain the second elastic body; and
a distance between the second groove and a base end of the second insertion tube portion is greater than a distance between the first groove and a base end of the first insertion tube portion.

6. The measuring device according to claim 1 wherein
the main body further comprises a main body recess which is open above the main body;
the measurement gauge further comprises a measurement insertion portion which is inserted into the main body recess;
the main body connection port is arranged at the main body recess;
the measurement connection port is arranged at the measurement insertion portion; and
the main body recess comprises
a pair of first sandwiching members which sandwich the measurement insertion portion in a first horizontal direction, and
a pair of second sandwiching members which sandwich the measurement insertion portion in a second horizontal direction that is perpendicular to the first horizontal direction.

7. The measuring device according to claim 6 wherein
the main body further comprises
an interlocking portion that abuts on and stops the measurement insertion portion from above, and
a displacing portion that causes the interlocking portion to be displaced between a locked position at which the measurement insertion portion is abutted on and stopped by the interlocking portion, and an unlocked position at which said abutment and stopping does not occur.

8. The measuring device according to claim 6 wherein
the measurement gauge further comprises a grip region that is capable of being gripped and that is arranged at a region which is at a top end and end at a first side in a first horizontal direction of the measurement gauge;
the main body comprises
an interlocking member that is displaced between a locked position at which an end at a first side in the first horizontal direction of the measurement insertion portion is abutted on and stopped from above, and an unlocked position at which said abutment and stopping does not occur; and
a locking force providing member which exerts an elastic restoring force on the interlocking member so as to cause the interlocking member to be located in the locked position; and
that first sandwiching member which of the pair of first sandwiching members is arranged at a second side in the first horizontal direction comprises
a lower sandwiching portion that opposes a region which is at a lower portion and end at a second side in the first horizontal direction of the measurement insertion portion; and
an actuation member which when actuated, is capable of displacing between a sandwiched position at which the actuation member abuts an end at a second side in the first horizontal direction of the measurement insertion portion above the lower sandwiching portion, and a nonsandwiched position at which the actuation member is separated from the measurement insertion portion.

9. The measuring device according to claim 6 wherein
the main body recess comprises a main body protrusion which protrudes in the first horizontal direction from the first sandwiching member;
a bottom end of the main body protrusion is arranged below a center in a vertical direction of the main body recess; and
a height with which the main body protrusion protrudes increases as one proceeds downward.

10. The measuring device according to claim 6 wherein
the measurement insertion portion comprises a measurement protrusion which protrudes in the first horizontal direction;
a top end of the measurement protrusion is arranged above a center in a vertical direction of the measurement insertion portion; and
a height with which the measurement protrusion protrudes increases as one proceeds upward.

11. A measuring system comprising:
the measuring device according to claim 1; and
a communication apparatus that is capable of communicating with the measuring device.

12. The measuring device according to claim 2 wherein a distance between the first groove and a tip end of the first insertion tube portion is less than a dimension in a tube axial direction of the first groove.

13. The measuring device according to claim 3 wherein a distance between the second groove and a tip end of the second insertion tube portion is less than a dimension in a tube axial direction of the second groove.

14. The measuring device according to claim 9 wherein
the measurement insertion portion comprises a measurement protrusion which protrudes in the first horizontal direction;
a top end of the measurement protrusion is arranged above a center in a vertical direction of the measurement insertion portion; and
a height with which the measurement protrusion protrudes increases as one proceeds upward.

15. The measuring device according to claim 14 wherein a bottom end of the measurement protrusion is arranged below a top end of the main body protrusion.

16. The measuring device according to claim 4 wherein
the connecting tube comprises
a first insertion tube portion which is arranged at the first end of the connecting tube and which is inserted into the interior of the measurement connection port, and
a second insertion tube portion which is arranged at the second end of the connecting tube and which is inserted into the interior of the main body connection port; and
a dimension in a tube axial direction of the second insertion tube portion is greater than a dimension in a tube axial direction of the first insertion tube portion.

17. The measuring device according to claim 4 wherein
the connecting tube comprises
a first insertion tube portion which is arranged at the first end of the connecting tube and which is inserted into the interior of the measurement connection port, and
a second insertion tube portion which is arranged at the second end of the connecting tube and which is inserted into the interior of the main body connection port; and
a maximum outside diameter of the first insertion tube portion is greater than a maximum outside diameter of the second insertion tube portion.

18. The measuring device according to claim 4 wherein an outside diameter of the first elastic body is greater than an outside diameter of the second elastic body.

19. The measuring device according to claim 1 wherein
the main body comprises a main body contact;
the measurement gauge comprises a measurement contact; and
when the measurement gauge is installed with respect to the main body, the measurement contact is made to come in contact with and be electrically connected to the main body contact.

20. The measuring device according to claim 19 wherein the measurement contact and the main body contact are arranged so as to be above the connecting tube when in a state in which the measurement gauge is installed with respect to the main body.
